(19) United States
(12) Patent Application Publication
(10) Pub. No.: US 2024/0299593 A1
(43) Pub. Date: Sep. 12, 2024

(54) SYSTEMS AND METHODS FOR GENERATION OF HYPERPOLARIZED MATERIALS

(71) Applicant: NVISION IMAGING TECHNOLOGIES GMBH, Ulm (DE)

(72) Inventors: Ilai SCHWARTZ, Neu-Ulm (DE); Michael KEIM, Neu-Ulm (DE); Stephan KNECHT, Stuttgart (DE); Christophoros VASSILIOU, Ulm (DE); Felix JOSTEN, Ulm (DE); Senay KARAALI, Ulm (DE)

(73) Assignee: NVISION IMAGING TECHNOLOGIES GMBH, Ulm (DE)

(21) Appl. No.: 18/573,662

(22) PCT Filed: Jun. 22, 2022
(86) PCT No.: PCT/IB2022/000346

§ 371 Date: Dec. 22, 2023

Related U.S. Application Data
(60) Provisional application No. 63/260,631, filed on Aug. 27, 2021.
(60) Provisional application No. 63/260,934, filed on Sep. 7, 2021.
(60) Provisional application No. 63/202,709, filed on Jun. 22, 2021.

(51) Int. Cl. A61K 49/06; A61K 9/08; B01J 19/08; G01R 33/28

(52) U.S. Cl. CPC A61K 49/06; A61K 9/08; B01J 19/087; G01R 33/282; B01J 2219/085

(57) ABSTRACT

The present disclosure describes hyperpolarized materials for use in nuclear magnetic resonance, magnetic resonance imaging, or similar applications. The present disclosure describes methods for producing hyperpolarized materials for use in nuclear magnetic resonance, magnetic resonance imaging, or similar applications. The present disclosure describes precursor compounds for use in producing hyperpolarized materials for use in nuclear magnetic resonance, magnetic resonance imaging, or similar applications.

### CROSS-REFERENCES TO RELATED APPLICATIONS

This application claims priority to: U.S. Provisional Patent Application No. 63/202,709, filed on Jun. 22, 2021; U.S. Provisional Patent Application No. 63/260,631, filed on Aug. 27, 2021; and U.S. Provisional Patent Application No. 63/260,934, filed on Sep. 7, 2021; each of which is incorporated herein by reference in its entirety for all purposes.

### TECHNICAL FIELD

The disclosed embodiments generally relate to the generation and purification of hyperpolarized materials for use in nuclear magnetic resonance, magnetic resonance imaging, or similar applications.

### BACKGROUND

Parahydrogen induced polarization (PHIP) is a method for polarizing metabolites for hyperpolarized (HP) Magnetic Resonance Imaging (MRI), with low cost and high throughput. Parahydrogen induced polarization with sidearm hydrogenation (PHIP-SAH) can be used to polarize metabolites, e.g., acetate molecules. However, existing PHIP-SAH polarization approaches may be unsuitable for preclinical or clinical HP MRI applications.

### SUMMARY

In some embodiments, the present disclosure describes methods for producing a dosage composition comprising a hyperpolarized biorelevant imaging agent or a pharmaceutically acceptable salt thereof. In some embodiments, the present disclosure describes methods for producing a dosage composition comprising a hyperpolarized biorelevant imaging agent or a pharmaceutically acceptable salt thereof, wherein the method comprises: —obtaining a solution in a vessel, wherein the solution comprises a first organic solvent, an aqueous mixture, the hyperpolarized biorelevant imaging agent or a pharmaceutically acceptable salt thereof, and optionally an unbonded sidearm; —one or more washing steps, wherein the solution is washed with a second organic solvent, thereby forming: (i) an organic mixture phase comprising the first organic solvent, the second organic solvent, and the optional unbonded sidearm, and (ii) an aqueous mixture phase comprising the aqueous mixture and the hyperpolarized biorelevant imaging agent or a pharmaceutically acceptable salt thereof; —one or more separation steps wherein the organic mixture phase is separated from the aqueous mixture phase, and either the organic mixture phase or the aqueous mixture phase is transferred into a separate vessel; and—obtaining a dosage composition from the aqueous mixture which comprises the hyperpolarized biorelevant imaging agent or a pharmaceutically acceptable salt thereof. In some embodiments, the method further comprises one or more evaporation steps, wherein the aqueous mixture phase is subjected to organic vapor extraction conditions to evaporate at least a portion of organic solvent remaining in the aqueous mixture. In some embodiments, the organic vapor extraction conditions comprise bubbling with an inert gas, such as nitrogen gas.

In some embodiments, the present disclosure describes methods for producing a dosage composition comprising a hyperpolarized biorelevant imaging agent or a pharmaceutically acceptable salt thereof, wherein the method comprises: —obtaining a solution in a vessel, wherein the solution comprises a first organic solvent, an aqueous mixture, the hyperpolarized biorelevant imaging agent or a pharmaceutically acceptable salt thereof, and optionally an unbonded sidearm; —one or more evaporation steps, wherein the solution is subjected to organic vapor extraction conditions to evaporate a portion of organic solvent, and optional unbonded sidearm, from the solution, thereby providing an aqueous mixture phase comprising the aqueous mixture and the hyperpolarized biorelevant imaging agent or a pharmaceutically acceptable salt thereof; and—obtaining a dosage composition from the aqueous mixture which comprises the hyperpolarized biorelevant imaging agent or a pharmaceutically acceptable salt thereof. In some embodiments, the organic vapor extraction conditions comprise bubbling with an inert gas, such as nitrogen gas. In some embodiments, the method further comprises: —one or more washing steps, wherein the solution or mixture is washed with a second organic solvent, thereby forming: (i) an organic mixture phase comprising the first organic solvent, the second organic solvent, and optional unbonded sidearm, and (ii) an aqueous mixture phase comprising the aqueous mixture and the hyperpolarized biorelevant imaging agent or a pharmaceutically acceptable salt thereof; and—one or more separation steps, wherein the organic mixture phase is separated from the aqueous mixture phase, and either the organic mixture phase or the aqueous mixture phase is transferred into a separate vessel.

In some embodiments, the methods of the present disclosure comprise a step of obtaining a solution in a vessel. In some embodiments, the step of obtaining a solution in a vessel comprises the following steps: —obtaining a solution comprising the first organic solvent and a biorelevant imaging agent precursor dissolved in the first organic solvent, wherein the biorelevant imaging agent precursor comprises: (i) a biorelevant imaging agent and a (ii) sidearm comprising an unsaturated carbon-carbon double bond (—C═C—) or carbon-carbon triple bond (—C≡C—); —hydrogenating the unsaturated carbon-carbon double bond (—C═C—) or carbon-carbon triple bond (—C≡C—) of the sidearm with parahydrogen in the first organic solvent, thereby forming a parahydrogenated derivative of the biorelevant imaging agent precursor; —applying a polarization transferring waveform to transfer nuclear spin order from the parahydrogen on the sidearm to a non-hydrogen nuclear spin on the biorelevant imaging agent; —optionally, hydrolyzing the parahydrogenated biorelevant imaging agent precursor by adding an aqueous hydrolyzing agent to the solution to produce the hyperpolarized biorelevant imaging agent and the unbonded sidearm; and—optionally neutralizing the solution with a buffer to slow or terminate the hydrolysis reaction; thereby producing the solution comprising the first organic solvent, the aqueous mixture, the hyperpolarized biorelevant imaging agent or a pharmaceutically acceptable salt thereof, and the optional unbonded sidearm (if the optional hydrolysis step was completed). In some embodiments, the step of obtaining a solution in a vessel further comprises a catalyst scavenging step prior to the optional additional of the aqueous hydrolyzing agent, wherein the catalyst scavenging step comprises filtering the solution to remove residual catalyst atoms (e.g., catalyst atoms remaining from the hydrogenation step), such as rhodium atoms, iridium atoms, and/or any other catalyst atoms from the solution. In some embodiments, the solution is concentrated (e.g., by evaporation) prior to the catalyst scavenging step.

In some embodiments, the biorelevant imaging agent precursor is a precursor comprising a compound of Formula Ia or Formula Ib. In some embodiments, the biorelevant imaging agent is selected from: pyruvate, glutamate, glutamine, lactate, acetate, acetoacetate, zymonate, alanine, fructose, fumarate, bicarbonate, urea, dehydroascorbate, alpha-ketoglutarate, dihydroxyacetone, glucose, ascorbate, and conjugate acids thereof. In some embodiments, the biorelevant imaging agent has a solubility in the first organic solvent of less than 50 millimolar (mM). In some embodiments, the biorelevant imaging agent has a solubility in water of greater than 50 millimolar (mM).

In some embodiments, the aqueous mixture comprises: water, sodium hydroxide, potassium hydroxide, or any mixture thereof.

In some embodiments, the first organic solvent comprises acetone, ethanol, methanol, chloroform, ethyl acetate, methyl ethyl ketone (MEK), acetophenone, hexone, cyclohexanone, cyclopentanone, or a combination thereof. In some embodiments, the first organic solvent comprises acetone. In some embodiments, the first organic solvent comprises methyl ethyl ketone (MEK). In some embodiments, the first organic solvent has a solubility in water of greater than 75 millimolar (mM) at 20° C. In some embodiments, the first organic solvent has a solubility in water at 20° C. which is greater than the solubility of chloroform in water at 20° C. In some embodiments, the first organic solvent does not comprise chloroform.

In some embodiments, the second organic solvent comprises one or more ICH class 2 solvents selected from: acetonitrile, chlorobenzene, chloroform, cyclohexane, dibromomethane (DBM), 1,2-dichloroethene, dichloromethane (DCM), 1,2-dimethoxyethane, n,n-dimethylacetamide, n,n-dimethylformamide, 1,4-dioxane, 2-ethoxyethanol, ethyleneglycol, formamide, hexane, methanol, 2-methoxyethanol, methylbutyl ketone, methylcyclohexane, n-methylpyrrolidone, nitromethane, pyridine, sulfolane, tetrahydrofuran, tetralin, toluene, 1,1,2-trichloroethene, or xylene. In some embodiments, the second organic solvent comprises one or more ICH class 3 solvents selected from: acetic acid, acetone, anisole, 1-butanol, 2-butanol, butyl acetate, methyl-tert-butyl ether (MTBE), cumene, diethyl ether, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, or propyl acetate. In some embodiments, the second organic solvent comprises methyl-tert-butyl ether (MTBE). In some embodiments, the second organic solvent comprises dibromomethane (DBM). In some embodiments, the second organic solvent comprises dichloromethane (DCM).

In some embodiments, the washing step and/or evaporation step is repeated until the first organic solvent has a concentration of 5000 ppm or less in the solution. In some embodiments, the washing step and/or evaporation step is repeated until the second organic solvent has a concentration in the solution below the ICH toxicity limit (e.g., see Table 1). In some embodiments, the present disclosure describes a dosage composition comprising hyperpolarized biorelevant imaging agent or a pharmaceutically acceptable salt thereof. In some embodiments, the present disclosure describes a dosage composition comprising hyperpolarized biorelevant imaging agent or a pharmaceutically acceptable salt thereof, wherein the dosage composition is produced by a method of the present disclosure. In some embodiments, the dosage composition comprises no more than 20 mM, 10 mM, 5 mM, 2 mM, or 1 mM of the first organic solvent. In some embodiments, the dosage composition comprises no more than 20 mM, 10 mM, 5 mM, 2 mM, or 1 mM of the second organic solvent.

In some embodiments, the present disclosure describes a system for implementing the methods of the present disclosure. In some embodiments, the present disclosure describes a system for implementing the methods of the present disclosure, wherein the system comprises a first vessel, and a second vessel fluidly connected with the first vessel. In some embodiments, the present disclosure describes a system for implementing the methods of the present disclosure, wherein the system comprises a first vessel, and a second vessel fluidly connected with the first vessel through a first fluid transfer element. In some embodiments, the present disclosure describes a system for implementing the methods of the present disclosure, wherein the system comprises a first vessel, a second vessel fluidly connected with the first vessel, and a third vessel fluidly connected with the second vessel. In some embodiments, the present disclosure describes a system for implementing the methods of the present disclosure, wherein the system comprises a first vessel, a second vessel fluidly connected with the first vessel through a first fluid transfer element, and a third vessel fluidly connected with the second vessel through a second fluid transfer element.

In some embodiments, the system further comprises a magnetic guide system. In some embodiments, the system further comprises a magnetic guide system which provides a defined magnetic field over at least a portion of the system. In some embodiments, the system further comprises a magnetic guide system which comprises one or more solenoids which provide the defined magnetic field. In some embodiments, the system further comprises a magnetic guide system which provides a defined magnetic field over the first vessel, second vessel, and/or optional third vessel. In some embodiments, the system further comprises a magnetic guide system which provides a defined magnetic field over the first fluid transfer element and/or optional second fluid transfer element. In some embodiments, the system further comprises a magnetic guide system which provides a defined magnetic field over the entire system. In some embodiments, the defined magnetic field prevents undesired hyperpolarization loss in the solution during implementation of the methods of the present disclosure.

### BRIEF DESCRIPTION OF THE DRAWINGS

The accompanying drawings, which comprise a part of this specification, illustrate several embodiments and, together with the description, serve to explain certain principles and features of the disclosed embodiments. In the drawings:

FIG. 1 depicts an embodiment 100 of a method for producing a dosage composition comprising a hyperpolarized biorelevant imaging agent, according to the present disclosure.

FIG. 2 depicts an embodiment 200 of a method for producing a dosage composition comprising a hyperpolarized biorelevant imaging agent, according to the present disclosure.

FIG. 3 depicts an embodiment 300 of a system for producing a dosage composition comprising a hyperpolarized biorelevant imaging agent, according to the present disclosure.

FIG. 4 depicts an embodiment 400 of a system for producing a dosage composition comprising a hyperpolarized biorelevant imaging agent, according to the present disclosure.

FIG. 5 depicts an embodiment 500 of a system for producing a dosage composition comprising a hyperpolarized biorelevant imaging agent, according to the present disclosure.

FIG. 6 shows NMR spectrum results for a SABRE catalyst in acetone extraction study, according to certain embodiments of the present disclosure.

### DETAILED DESCRIPTION

Reference will now be made in detail to exemplary embodiments, discussed with regards to the accompanying drawings. Unless otherwise defined, technical and/or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

Recent work in the field of nuclear magnetic resonance (NMR) and magnetic resonance imaging (MRI) has demonstrated that NMR and MRI signals associated with a variety of biorelevant imaging agents can be enhanced by many orders of magnitude using a variety of so-called hyperpolarization techniques. This signal enhancement allows for improved spectroscopic analysis of the biorelevant imaging agent as it is metabolized by various tissues at different locations within a body. Analysis of the metabolic information determined by such spectroscopic imaging may allow for non-invasive determination of a health state of tissue within a body. For example, abnormal metabolism of a biorelevant imaging agent may be indicative of a disease such as cancer at some location in the body.

Existing techniques for hyperpolarizing biorelevant imaging agents include dissolution dynamic nuclear polarization (DNP), parahydrogen induced polarization (PHIP), PHIP-sidearm hydrogenation (PHIP-SAH), and signal amplification by reversible exchange (SABRE). In PHIP and PHIP-SAH, a precursor of the biorelevant imaging agent is reacted with parahydrogen to form a parahydrogenated derivative of the precursor. Spin order is then transferred from the protons added via the parahydrogenation reaction to a nucleus of interest (such as a carbon-13 nucleus) contained within the biorelevant imaging agent. In PHIP-SAH, the parahydrogenated derivative of the precursor is cleaved (e.g., hydrolyzed) to yield the hyperpolarized biorelevant imaging agent. The biorelevant imaging agent is then purified and used in an NMR or MRI procedure. In PHIP-SAH, the precursor can comprise a biorelevant imaging agent coupled to a sidearm containing at least one unsaturated bond (e.g., at least one carbon-carbon double bond or at least one carbon-carbon triple bond) suitable for reaction with parahydrogen. In SABRE, the biorelevant imaging agent itself is directly hyperpolarized (i.e., without the need for a parahydrogenation reaction) via the temporary formation of a complex between the biorelevant imaging agent, parahydrogen, and a polarization transfer catalyst. However, previous methods for producing biorelevant imaging agents either insufficient polarization levels or concentrations for clinical relevance (e.g., when performing all or part of the SABRE, PHIP, and/or PHIP-SAH process in aqueous solution) or place the biorelevant imaging agents in mixtures that contain excessive concentrations of harmful organic solvents (e.g., when performing all or part of the SABRE, PHIP, and/or PHIP-SAH process in organic solvents). Accordingly, there is a need for new methods for producing biorelevant imaging agents that achieve clinically relevant polarizations, concentrations, volumes, and purity.

The disclosed embodiments include systems and methods for producing biorelevant imaging agents, in clinically relevant polarizations, concentrations, volumes, and purity. Disclosed embodiments provide technical improvements in polarizing biorelevant imaging agents in solution. These technical improvements support increases in biorelevant imaging agent concentration and the degree of biorelevant polarization.

### Hyperpolarization and Parahydrogen

As used in the present disclosure, hyperpolarization describes a condition in which an absolute value of a difference between a population of spin states (e.g., nuclear spin states, proton spin states, or the like) being in one state (e.g., spin up) and a population of a spin states being in another state (e.g., spin down) exceeds the absolute value of the corresponding difference at thermal equilibrium.

Parahydrogen can be used as a source of polarization, consistent with disclosed embodiments. Parahydrogen, as described herein, is a form of molecular hydrogen in which the two proton spins are in the singlet state. The disclosed embodiments are not limited to a particular method of generating parahydrogen. Parahydrogen may be formed in a gas form or in a liquid form. In some embodiments, parahydrogen is generated in gas form by flowing hydrogen gas at low temperature through a chamber with a catalyst (e.g., iron oxide or another suitable catalyst). The hydrogen gas can contain both parahydrogen and orthohydrogen. The low temperature can bring the hydrogen gas to thermodynamic equilibrium in the chamber, increasing the population of parahydrogen.

The disclosed embodiments are not limited to a particular parahydrogen generation location or use location. Parahydrogen can be generated at a first location and subsequently transported to a second location for use. In some embodiments, the first location is a chamber, which may be part of a container, bottle, holder or other regions capable of holding a gas or a liquid. Such a chamber may be maintained at a suitable pressure or temperature. In some embodiments, the first location is a physical location such as a room, a lab, a particular warehouse, hospital or other location where the parahydrogen is generated.

The disclosed embodiments are not limited to a particular parahydrogen transport method. The generated parahydrogen may be transported in a chamber, which may be different from the chamber where the parahydrogen was generated. The chamber transporting the parahydrogen gas may be maintained at a suitable pressure or temperature, which may be transported by vehicle or persons. Transporting the parahydrogen may involve moving the parahydrogen from one container to a different container. Transporting the parahydrogen may involve moving the parahydrogen within the same location, such as from one part of a room to another part of the room. Transporting the parahydrogen may involve moving the parahydrogen from one room in a building to a different room in the same building or to a nearby building. Transporting the parahydrogen may involve moving the parahydrogen to a different location in another part of the same city, or a different city. Transporting the parahydrogen may involve bringing the parahydrogen into the vicinity of a polarizer, an NMR device, or an MRI device. Transporting the parahydrogen may involve packaging or shipping the parahydrogen in suitable containers.

In some embodiments, a population difference between two spin states is the difference between the population of the two spin states divided by the total population of the two spin states. A population difference may be expressed as a fractional population difference or a percentage population difference. In some embodiments, the fractional population difference is at least about 0.1, 0.2, 0.3, 0.4, 0.5, 0.6, 0.7, 0.8, 0.9, or more, at most about 0.9, 0.8, 0.7, 0.6, 0.5, 0.4, 0.3, 0.2, 0.1, or less, or within a range defined by any two of the preceding values.

Hydrogen gas can exhibit a population difference between proton spin states which greatly exceeds the population difference between proton spin states at thermal equilibrium. Parahydrogen can have a large population difference between the singlet spin state and any of the triplet spin states. In the case of Iz1Iz2, there is a large population difference, for example, between the spin state |↑>|↓> and the spin state |↑>>|↑|>. The population difference in proton spin states can be at least about 0.1 (e.g., a 10% difference in spin states-55% of the parahydrogen molecules in a sample being in the singlet state and 45% in the triplet state), 0.2, 0.3, 0.4, 0.5, 0.6, 0.7, 0.8, 0.9, or more, at most about 0.9, 0.8, 0.7, 0.6, 0.5, 0.4, 0.3, 0.2, 0.1, or less, or within a range defined by any two of the preceding values.

### Biorelevant Imaging Agents

The disclosed embodiments include systems and methods for producing and utilizing biorelevant imaging agents with clinically relevant polarizations, concentrations, volumes, or purities. In some embodiments, the method is for preparing an NMR material. In some embodiments, the NMR material is suitable for use in NMR or MRI operations. In some embodiments, the NMR material increases NMR or MRI signal and signal-to-noise ratio (SNR). In some embodiments, the NMR material is suitable for use in solution NMR spectroscopy. In some embodiments, the NMR material is a chemical compound. In some embodiments, the NMR material is a metabolite (e.g., a molecule with a biological relevance such as an amino acid, a saccharide, a derivative thereof, or the like), such as a metabolite suitable for use in an NMR metabolomics application. In some embodiments, the NMR material is suitable for in-vitro probing of the metabolism of a cell culture or other biological tissue. In some embodiments, the NMR material is used in an NMR probe to investigate a transient effect in which high signal enhancement due to hyperpolarization is needed, such as proton exchange between water and biomolecules. In some embodiments, the NMR material is a small molecule or metabolite suitable for injection into a cell, tissue or organism for detection in an MRI scan. In some embodiments, the NMR material is introduced into a chamber for further analysis by NMR or MRI operations. In some embodiments, the NMR material is enriched with one or more deuterium (²H) or carbon-13 (¹³C) atoms.

Consistent with disclosed embodiments, NMR material can include biorelevant imaging agents. In some embodiments, the biorelevant imaging agent can be suitable for use in NMR or MRI operations. In some embodiments, the biorelevant imaging agent may increase NMR or MRI signal or signal-to-noise ratio (SNR). In some embodiments, the biorelevant imaging agent can be suitable for use in solution NMR spectroscopy. In some embodiments, the biorelevant imaging agent may be a metabolite (e.g., a molecule with a biological relevance such as an amino acid, a saccharide, a derivative thereof, or the like), such as a metabolite suitable for use in an NMR metabolomics application. In some embodiments the biorelevant imaging agent is used for perfusion imaging or contrast enhanced imaging in MRI scans. In some embodiments, the biorelevant imaging agent is suitable for in-vitro probing of the metabolism of a cell culture or other biological tissue. In some embodiments, the biorelevant imaging agent is used for in-vitro probing of the metabolism of a cell culture or other biological tissue. In some embodiments, the biorelevant imaging agent is used in an NMR probe to investigate a transient effect in which high signal enhancement due to hyperpolarization is needed, such as proton exchange between water and biomolecules. In some embodiments, the biorelevant imaging agent is a small molecule or metabolite suitable for injection into a cell, tissue or organism for detection in an MRI scan. In some embodiments, the biorelevant imaging agent is introduced into a chamber for further analysis by NMR or MRI operations. In some embodiments, the biorelevant imaging agent is enriched with one or more ²H or ¹³C atoms.

In some embodiments, the biorelevant imaging agent comprises pyruvate, lactate, alpha-ketoglutarate, bicarbonate, fumarate, urea, dehydroascorbate, glutamate, glutamine, acetate, dihydroxyacetone, acetoacetate, glucose, ascorbate, zymonate, alanine, fructose, imidazole, nicotinamide, nitroimidazole, pyrazinamide, isoniazid, a conjugate acid of any of the foregoing, natural and unnatural amino acids, esters thereof, or ²H, ¹³C, or nitrogen-15 (¹⁵N) enriched versions of any of the foregoing. In some embodiments, the biorelevant imaging agent comprises pyruvate, lactate, alpha-ketoglutarate. In some embodiments, the biorelevant imaging agent comprises pyruvate. In some embodiments, the biorelevant imaging agent comprises lactate. In some embodiments, the biorelevant imaging agent comprises alpha-ketoglutarate (e.g., ethyl alpha-ketoglutarate).

In some embodiments, the biorelevant imaging agent comprises at least one non-hydrogen nuclear spin. In some embodiments, the non-hydrogen nuclear comprises at least one spin-1/2 atom. In some embodiments, the non-hydrogen nuclear spin comprises ¹³C or ¹⁵N. In some embodiments, the biorelevant imaging agent is at least partially isotopically labeled with the non-hydrogen nuclear spin. In some embodiments, the biorelevant imaging agent is at least partially enriched with the non-hydrogen nuclear spin when compared to an analog of the biorelevant imaging agent that features the non-hydrogen nuclear spin at its natural abundance. In some embodiments, the biorelevant imaging agent is enriched to feature the non-hydrogen nuclear spin at an abundance of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more, at most about 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less, or an abundance that is within a range defined by any two of the preceding values.

In some embodiments, the non-hydrogen nuclear spin replaces an NMR-inactive (i.e., spin-0) nucleus (e.g., ¹²C or a quadrupolar (i.e., spin >1/2) nucleus (e.g., nitrogen-14, ¹⁴N) of the analog of the biorelevant imaging agent that features the non-hydrogen nuclear spin at its natural abundance. For example, an analog of pyruvate that features ¹³C at its natural abundance may include about 98.9%¹²C and about 1.1% ¹³C at either C* in the structure H₃C—C*(═O)—C*OOH. As a biorelevant imaging agent, pyruvate may instead be isotopically enriched with ¹³C such that one or both C* comprises ¹³C at any abundance described herein. As used herein, *C and C* describe a carbon that can be either a ¹²C or ¹³C carbon isotope. As another example, an analog of urea that features ¹⁵N at its natural abundance may include about 99.6% ¹⁴N and about 0.4% ¹⁵N at either N* in the structure H₂N*—C(═O)—*NH₂. As a biorelevant imaging agent, urea may instead be isotopically enriched with ¹⁵N such that one or both N* comprises ¹⁵N at any abundance described herein. As used herein, *N and N* describe a nitrogen that can be either a ¹⁴N or ¹⁵N nitrogen isotope.

### Biorelevant Imaging Agent Precursors

In some embodiments, the present disclosure describes precursors (i.e., precursor compounds) which comprise a biorelevant imaging agent and a sidearm. In some embodiments, the biorelevant imaging agent is covalently attached to the sidearm. In some embodiments, the biorelevant imaging agent is attached to the sidearm through a transfer moiety, such as a PHIP transfer moiety, which is part of the sidearm.

In some embodiments, the present disclosure describes precursors (i.e., precursor compounds) which comprise an acyl derivative of a biorelevant imaging agent (i.e., R—C(═O)—) and a sidearm. As used herein, the term “acyl derivative of a biorelevant imaging agent” refers to a covalently-bonded derivative of a biorelevant imaging agent in which a terminal acid moiety [R—C(═O)OH)] of an unbonded biorelevant imaging agent is altered to an acyl group and covalent bond [R—C(═O)—)] in the bonded biorelevant imaging agent. In some embodiments, the acyl derivative of the biorelevant imaging agent is covalently attached to the sidearm. In some embodiments, the acyl derivative of the biorelevant imaging agent is attached to the sidearm through a transfer moiety, such as a PHIP transfer moiety, which is part of the sidearm.

The sidearm can be parahydrogenated using parahydrogen (e.g., by mixing the precursor and the parahydrogen). In some embodiments, the hydrogenation creates Iz1Iz2 order, the lower energy state between |↑>|↓|>, |↓|>|↑> or singlet spin order on two hydrogens spins, depending on whether the hydrogenation is performed at a low magnetic field or high magnetic field.

In some embodiments, the precursor is chosen such that, following hydrogenation and other optional chemical reactions, the biorelevant imaging agent is suitable for use in hyperpolarized NMR or MRI applications. In some embodiments, additional chemical reactions following hydrogenation can be used to separate the biorelevant imaging agent from the precursor. Such additional chemical reactions may include cleaving the sidearm of the precursor, e.g., by hydrolysis. For example, the biorelevant imaging agent can be a metabolite molecule, such that the precursor can a derivative of the metabolite molecule, with the derivative having the generic chemical structure of Formula Ia or Formula Ib. The biorelevant imaging agent can be polarized using the PHIP-SAH method (i.e., parahydrogenation of the sidearm and subsequent polarization transfer to the biorelevant imaging agent). Following hydrogenation and polarization transfer, the linking bond in the precursor (e.g., ester bond) may be hydrolyzed to produce a polarized biorelevant imaging agent and a separate sidearm element.

As used herein, hydrolysis is defined as the cleavage of a molecule via a nucleophilic substitution reaction, with the addition of the elements of water. Hydrolysis can be also performed under anhydrous conditions in the presence of hydroxide ions.

Consistent with disclosed embodiments, precursors of the general chemical form presented in Formula Ia or Formula Ib can be used as precursors for PHIP-SAH. Following hydrogenation of such precursors, the two 1H spins exhibiting the spin order are near (e.g., only three, four, or five bonds away) from the target carbon or nitrogen on the metabolite, which can be ¹³C enriched or ¹⁵N enriched as described herein. In some embodiments, a high J-coupling between the ¹³C or ¹⁵N spin and at least one of the ¹H spins derived from parahydrogen is achieved. In some embodiments, a J-coupling is achieved of at least about 0.1 hertz (Hz), 0.2 Hz, 0.3 Hz, 0.4 Hz, 0.5 Hz, 0.6 Hz, 0.7 Hz, 0.8 Hz, 0.9 Hz, 1 Hz, 2 Hz, 3 Hz, 4 Hz, 5 Hz, 6 Hz, 7 Hz, 8 Hz, 9 Hz, 10 Hz, or more, at most about 10 Hz, 9 Hz, 8 Hz, 7 Hz, 6 Hz, 5 Hz, 4 Hz, 3 Hz, 2 Hz, 1 Hz, 0.9 Hz, 0.8 Hz, 0.7 Hz, 0.6 Hz, 0.5 Hz, 0.4 Hz, 0.3 Hz, 0.2 Hz, 0.1 Hz, or less, or a J-coupling that is within a range defined by any two of the preceding values. For instance, in some embodiments, the J-coupling is between 1 Hz and 2 Hz, between 1 Hz and 3 Hz, between 1 Hz and 4 Hz, between 1 Hz and 5 Hz, between 1 Hz and 6 Hz, between 1 Hz and 7 Hz, between 1 Hz and 8 Hz, between 1 Hz and 9 Hz, between 1 Hz and 10 Hz, between 2 Hz and 3 Hz, between 2 Hz and 4 Hz, between 2 Hz and 5 Hz, between 2 Hz and 6 Hz, between 2 Hz and 7 Hz, between 2 Hz and 8 Hz, between 2 Hz and 9 Hz, between 2 Hz and 10 Hz, between 3 Hz and 4 Hz, between 3 Hz and 5 Hz, between 3 Hz and 6 Hz, between 3 Hz and 7 Hz, between 3 Hz and 8 Hz, between 3 Hz and 9 Hz, between 3 Hz and 10 Hz, between 4 Hz and 5 Hz, between 4 Hz and 6 Hz, between 4 Hz and 7 Hz, between 4 Hz and 8 Hz, between 4 Hz and 9 Hz, between 4 Hz and 10 Hz, between 5 Hz and 6 Hz, between 5 Hz and 7 Hz, between 5 Hz and 8 Hz, between 5 Hz and 9 Hz, between 5 Hz and 10 Hz, between 6 Hz and 7 Hz, between 6 Hz and 8 Hz, between 6 Hz and 9 Hz, between 6 Hz and 10 Hz, between 7 Hz and 8 Hz, between 7 Hz and 9 Hz, between 7 Hz and 10 Hz, between 8 Hz and 9 Hz, between 8 Hz and 10 Hz, or between 9 Hz and 10 Hz. Such a J-coupling may enable efficient polarization of the ¹³C spin.

Disclosed herein are novel precursors, including the compounds of Formulas Ia, Ib, IIa, IIb, IIIa, IIIb, IVa and IVb, tautomers thereof, deuterated derivatives of those compounds and their tautomers, salts thereof, and ¹³C or ¹⁵N enriched derivatives at one or more sites within the molecule (which may be in turn subject to hyperpolarization), and the subsequent generation of precursors given by the general Formulas Ia, Ib, IIa, II, IIIa, IIIb, IVa and IVb.

### Precursors of Formula Ia and Formula Ib

In some embodiments, the precursor comprises a compound of Formula Ia. Formula Ia encompasses the following structure:

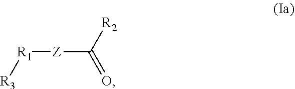

and includes tautomers thereof, deuterated derivatives of those compounds and their tautomers, pharmaceutically acceptable salts thereof, and ¹³C or ¹⁵N enriched derivatives at one or more sites. In some embodiments, Z of Formula Ia describes: (i) a carbon-carbon double bond (—C═C—) which is substituted to include ¹H (proton), ²H (deuterium), or a combination thereof (e.g., —C¹H═C¹H—, —C¹H═C²H—, —C²H═C²H—) or (ii) a carbon-carbon triple bond (—C≡C—). In some embodiments, R₁ of Formula Ia comprises a parahydrogen induced polarization (PHIP) transfer moiety, as described herein. In some embodiments, R₂ of Formula Ia comprises an optionally substituted hydrocarbon, alkoxy group, primary amine, secondary amine, or tertiary amine, as described herein. In some embodiments, R₃ of Formula Ia comprises a biorelevant imaging agent, as described herein. In Formula Ia, all moieties to the right of the R₃-R₄ bond (i.e., —R₁—Z—(C═O)—R₂) may be collectively referred to as a sidearm.

In some embodiments, the precursor comprises a compound of Formula Ib. Formula Ib encompasses the following structure:

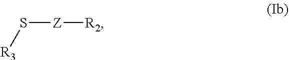

and includes tautomers thereof, deuterated derivatives of those compounds and their tautomers, pharmaceutically acceptable salts thereof, and ¹³C enriched derivatives at one or more sites. In some embodiments, Z in Formula Ib denotes an ethynyl (—C≡C—) group, an optionally substituted prop-2-ynyl (—C—C≡C—) group, an optionally substituted ethenyl (—C≡C—) group, an optionally substituted prop-2-enyl (—C—C≡C—) group, or an optionally substituted but-3-enyl (—C—C—C═C—) group. In some embodiments, R₂ in Formula Ib comprises an optionally substituted hydrocarbon group, alkyl group, cyclic alkyl group, aryl group, carboxyl group, keto group, or alkoxy group, as described herein. In some embodiments, R₃ in Formula Ib comprises an acyl derivative of a biorelevant imaging agent, as described herein. In Formula Ib, all moieties to the right of the R₃ group (i.e., —S—Z—R₂) may be collectively referred to as a sidearm.

In some embodiments, the compound of Formula Ia or Formula Ib has a solubility in water of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in water that is within a range defined by any two of the preceding values.

In some embodiments, the compound of Formula Ia or Formula Ib has a solubility in an organic solvent (e.g., acetone, ethanol, chloroform, toluene) of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in an organic solvent that is within a range defined by any two of the preceding values.

In some embodiments, the compound of Formula Ia comprises methyl 4-((2-oxopropanoyl)oxy)but-2-ynoate. In some embodiments, the compound of Formula Ia comprises methyl 4-((2-hydroxypropanoyl)oxy)but-2-ynoate. In some embodiments, the compound of Formula Ia comprises 5-((4-methoxy-4-oxobut-2-yn-1-yl)oxy)-4,5-dioxopentanoic acid.

In some embodiments, the compound of Formula Ia comprises isopropyl 4-((2-oxopropanoyl)oxy)but-2-ynoate. In some embodiments, the compound of Formula Ia comprises isopropyl 4-((2-hydroxypropanoyl)oxy)but-2-ynoate. In some embodiments, the compound of Formula Ia comprises 5-((4-isopropoxy-4-oxobut-2-yn-1-yl)oxy)-4,5-dioxopentanoic acid.

In some embodiments, the compound of Formula Ia comprises tert-butyl 4-((2-oxopropanoyl)oxy)but-2-ynoate. In some embodiments, the compound of Formula Ia comprises tert-butyl 4-((2-hydroxypropanoyl)oxy)but-2-ynoate. In some embodiments, the compound of Formula Ia comprises 5-((4-(tert-butoxy)-4-oxobut-2-yn-1-yl)oxy)-4,5-dioxopentanoic acid.

In some embodiments, the compound of Formula Ia comprises tert-butyl 4-((2-oxopropanoyl-1-¹³C)oxy)but-2-ynoate. In some embodiments, the compound of Formula Ia comprises tert-butyl 4-((2-hydroxypropanoy-1-¹³C l)oxy)but-2-ynoate. In some embodiments, the compound of Formula Ia comprises 5-((4-(tert-butoxy)-4-oxobut-2-yn-1-yl)oxy)-4,5-dioxopentanoic ¹³C acid.

In some embodiments, the compound of Formula Ia comprises 2-(Methyl-d3)propan-2-yl-1,1,1,3,3,3-d6 4-((2-oxopropanoyl-1-¹³C)oxy)but-2-ynoate. In some embodiments, the compound of Formula Ia comprises 2-(Methyl-d3)propan-2-yl-1,1,1,3,3,3-d6 4-((2-hydroxypropanoyl-1-¹³C)oxy)but-2-ynoate. In some embodiments, the compound of Formula Ia comprises 5-((4-((2-(methyl-d3)propan-2-yl-1,1,1,3,3,3-d6)oxy)-4-oxobut-2-yn-1-yl)oxy)-4,5-dioxopentanoic acid.

In some embodiments, the compound of Formula Ia comprises tert-butyl 4-((2-oxopropanoyl)oxy)but-2-ynoate-4-d. In some embodiments, the compound of Formula Ia comprises tert-butyl 4-((2-hydroxypropanoyl)oxy)but-2-ynoate-4-d. In some embodiments, the compound of Formula Ia comprises 5-((4-(tert-butoxy)-4-oxobut-2-yn-1-yl-1-d)oxy)-4,5-dioxopentanoic acid.

In some embodiments, the compound of Formula Ia comprises tert-butyl 4-((2-oxopropanoyl)oxy)pent-2-ynoate. In some embodiments, the compound of Formula Ia comprises tert-butyl 4-((2-hydroxypropanoyl)oxy)pent-2-ynoate. In some embodiments, the compound of Formula Ia comprises 5-((5-(tert-butoxy)-5-oxopent-3-yn-2-yl)oxy)-4,5-dioxopentanoic acid.

In some embodiments, the compound of Formula Ia comprises tert-butyl 4-((2-oxopropanoyl)oxy)-4-phenylbut-2-ynoate. In some embodiments, the compound of Formula Ia comprises tert-butyl 4-((2-hydroxypropanoyl)oxy)-4-phenylbut-2-ynoate. In some embodiments, the compound of Formula Ia comprises 5-((4-(tert-butoxy)-4-oxo-1-phenylbut-2-yn-1-yl)oxy)-4,5-dioxopentanoic acid.

In some embodiments, the compound of Formula Ia comprises benzhydryl 4-((2-oxopropanoyl)oxy)but-2-ynoate. In some embodiments, the compound of Formula Ia comprises benzhydryl 4-((2-hydroxypropanoyl)oxy)but-2-ynoate. In some embodiments, the compound of Formula Ia comprises 5-((4-(benzhydryloxy)-4-oxobut-2-yn-1-yl)oxy)-4,5-dioxopentanoic acid.

In some embodiments, the compound of Formula Ia comprises 4-oxo-4-phenylbut-2-yn-1-yl 2-oxopropanoate. In some embodiments, the compound of Formula Ia comprises 4-oxo-4-phenylbut-2-yn-1-yl 2-hydroxypropanoate. In some embodiments, the compound of Formula Ia comprises 4,5-dioxo-5-((4-oxo-4-phenylbut-2-yn-1-yl)oxy)pentanoic acid.

In some embodiments, the compound of Formula Ia comprises 4-oxo-4-(phenyl-ds)but-2-yn-1-yl 2-oxopropanoate. In some embodiments, the compound of Formula Ia comprises 4-oxo-4-(phenyl-ds)but-2-yn-1-yl 2-hydroxypropanoate. In some embodiments, the compound of Formula Ia comprises 4,5-dioxo-5-((4-oxo-4-(phenyl-ds)but-2-yn-1-yl)oxy)pentanoic acid.

In some embodiments, the compound of Formula Ia comprises tert-butyl 4-acetoxybut-2-ynoate. In some embodiments, the compound of Formula Ia comprises 1-(4-(Tert-butoxy)-4-oxobut-2-yn-1-yl) 5-ethyl 2-oxopentanedioate.

In some embodiments, the compound of Formula Ia comprises Methyl 4-(2,2-dichloroacetoxy)but-2-ynoate.

In some embodiments, the compound of Formula Ia comprises trityl 4-((2-oxopropanoyl)oxy)but-2-ynoate. In some embodiments, the compound of Formula Ia comprises trityl 4-((2-hydroxypropanoyl)oxy)but-2-ynoate. In some embodiments, the compound of Formula Ia comprises 4,5-dioxo-5-((4-oxo-4-(trityloxy)but-2-yn-1-yl)oxy)pentanoic acid.

In some embodiments, the compound of Formula Ia comprises 4-(diphenylamino)-4-oxobut-2-yn-1-yl 2-oxopropanoate. In some embodiments, the compound of Formula Ia comprises 4-(diphenylamino)-4-oxobut-2-yn-1-yl 2-hydroxypropanoate. In some embodiments, the compound of Formula Ia comprises 5-((4-(diphenylamino)-4-oxobut-2-yn-1-yl)oxy)-4,5-dioxopentanoic acid.

In some embodiments, the compound of Formula Ia comprises 4-(diisopropylamino)-4-oxobut-2-yn-1-yl 2-oxopropanoate. In some embodiments, the compound of Formula Ia comprises 4-(diisopropylamino)-4-oxobut-2-yn-1-yl 2-hydroxypropanoate. In some embodiments, the compound of Formula Ia comprises 5-((4-(diisopropylamino)-4-oxobut-2-yn-1-yl)oxy)-4,5-dioxopentanoic acid.

In some embodiments, the compound of Formula Ia comprises 4-oxopent-2-yn-1-yl 2-oxopropanoate. In some embodiments, the compound of Formula Ia comprises 4-oxopent-2-yn-1-yl 2-hydroxypropanoate. In some embodiments, the compound of Formula Ia comprises 4,5-dioxo-5-((4-oxopent-2-yn-1-yl)oxy)pentanoic acid.

In some embodiments, the compound of Formula Ia comprises 4-oxo-4-(pyridin-2-yl)but-2-yn-1-yl 2-oxopropanoate. In some embodiments, the compound of Formula Ia comprises 4-oxo-4-(pyridin-2-yl)but-2-yn-1-yl 2-hydroxypropanoate. In some embodiments, the compound of Formula Ia comprises 4,5-dioxo-5-((4-oxo-4-(yridine-2-yl)but-2-yn-1-yl)oxy)pentanoic acid.

In some embodiments, the compound of Formula Ia comprises 4-(1-methyl-1H-imidazol-2-yl)-4-oxobut-2-yn-1-yl 2-oxopropanoate. In some embodiments, the compound of Formula Ia comprises 4-(1-methyl-1H-imidazol-2-yl)-4-oxobut-2-yn-1-yl 2-hydroxypropanoate. In some embodiments, the compound of Formula Ia comprises 5-((4-(1-methyl-1H-imidazol-2-yl)-4-oxobut-2-yn-1-yl)oxy)-4,5-dioxopentanoic acid.

In some embodiments, the composition of Formula Ib comprises S-(phenylethynyl) ethanethioate. In some embodiments, the composition of Formula Ib comprises S-(3-phenylprop-2-yn-1-yl) ethanethioate. In some embodiments, the composition of Formula Ib comprises S-(3-(4-methoxyphenyl)prop-2-yn-1-yl) ethanethioate. In some embodiments, the composition of Formula Ib comprises S-(3-(3,4,5-trimethoxyphenyl)prop-2-yn-1-yl) ethanethioate. In some embodiments, the composition of Formula Ib) comprises S-(3-(benzo[d][1,3]dioxol-5-yl)prop-2-yn-1-yl) ethanethioate. In some embodiments, the composition of Formula Ib comprises S-(3-(4-nitrophenyl)prop-2-yn-1-yl) ethanethioate. In some embodiments, the composition of Formula Ib comprises tert-butyl 4-(acetylthio)but-2-ynoate. In some embodiments, the composition of Formula Ib comprises tert-butyl 4-(2-oxopropanoyl)thio)but-2-ynoate. In some embodiments, the composition of Formula Ib comprises S-(3-phenylprop-2-yn-1-yl) 2-oxopropanethioate. In some embodiments, the composition of Formula Ib comprises S-(3-(4-methoxyphenyl)prop-2-yn-1-yl) 2-oxopropanethioate. In some embodiments, the composition of Formula Ib comprises S-(3-(3,4,5-trimethoxyphenyl)prop-2-yn-1-yl) 2-oxopropanethioate. In some embodiments, the composition of Formula Ib) comprises S-(3-(benzo[d][1,3]dioxol-5-yl)prop-2-yn-1-yl) 2-oxopropanethioate.

### Parahydrogenated Precursors of Formula IIa and Formula IIb

In some embodiments, the compound of Formula Ia is parahydrogenated (i.e., modified via the addition of parahydrogen protons across Z via a hydrogenation reaction between Formula Ia and a parahydrogen), as described herein. In some embodiments, parahydrogenation of a compound of Formula Ia yields a compound of Formula IIa. Formula IIa encompasses the following structure:

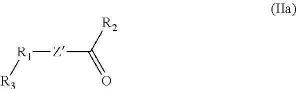

and includes tautomers thereof, deuterated derivatives of those compounds and their tautomers, pharmaceutically acceptable salts thereof, and ¹³C or ¹⁵N enriched derivatives at one or more sites. In some embodiments, Z′ of Formula IIa is: (i) a parahydrogenated carbon-carbon single bond (—CH*—CH*—) which is substituted to include ¹H (proton), ²H (deuterium), or a combination thereof (e.g., —CH₂H*—CH₂H*—, —CHDH*—CH₂H*—, —CD₂H*—CH₂H*—), or (ii) a parahydrogenated carbon-carbon double bond (—CH*═CH*—) which is substituted to include ¹H (proton), ²H (deuterium), or a combination thereof. In some embodiments, H* denotes a hydrogen having a spin order derived from parahydrogen (i.e., a hydrogen atom or proton added across the carbon-carbon double bond or the carbon-carbon triple bond Z via a hydrogenation reaction between a compound of Formula Ia and a parahydrogen, as described herein). In some embodiments, H* denotes a hydrogen having a spin order derived from parahydrogen (e.g., before polarization transfer). In some embodiments, R₁ of Formula IIa comprises a PHIP transfer moiety, as described herein. In some embodiments, R₂ of Formula IIa comprises an optionally substituted hydrocarbon, alkoxy group, primary amine, secondary amine, or tertiary amine, as described herein. In some embodiments, R₃ of Formula IIa comprises a biorelevant imaging agent, as described herein. In Formula IIa, all moieties to the right of the R₃—R₁ bond (i.e., —R₁—Z′—(C═O)—R₂) may be collectively referred to as a parahydrogenated sidearm.

In some embodiments, the compound of Formula Ib is parahydrogenated (i.e., modified via the addition of parahydrogen protons across Z via a hydrogenation reaction between Formula Ib and a parahydrogen), as described herein. In some embodiments, parahydrogenation of a compound of Formula Ib yields a compound of Formula IIb. Formula IIb encompasses the following structure:

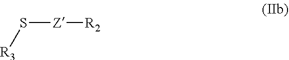

and includes tautomers thereof, deuterated derivatives of those compounds and their tautomers, pharmaceutically acceptable salts thereof, and ¹³C enriched derivatives at one or more sites. In some embodiments, Z′ of Formula IIb denotes a parahydrogenated ethenyl (—CH*═CH*—) group, an optionally substituted parahydrogenated prop-2-enyl (—C—CH*═CH*—) group, an optionally substituted parahydrogenated ethanyl (—CH*—CH*—) group, an optionally substituted parahydrogenated propanyl (—C—CH*—CH*—) group, or an optionally substituted parahydrogenated butanyl (—C—C—CH*═CH*—) group. In some embodiments, H* denotes a hydrogen having a spin order derived from parahydrogen (i.e., a hydrogen atom or proton added across the carbon-carbon double bond or the carbon-carbon triple bond Z via a hydrogenation reaction between a compound of Formula I and a parahydrogen, as described herein). In some embodiments, H* denotes a hydrogen having a spin order derived from parahydrogen (e.g., before polarization transfer). In some embodiments, R₂ of Formula IIb comprises an optionally substituted hydrocarbon group, alkyl group, cyclic alkyl group, aryl group, carboxyl group, keto group, or alkoxy group, as described herein. In some embodiments, R₃ of Formula IIb comprises an acyl derivative of a biorelevant imaging agent, as described herein. In Formula IIb, all moieties to the right of the R₃ group (i.e., S—Z′—R₂) may be collectively referred to as a parahydrogenated sidearm.

In some embodiments, the compound of Formula IIa or Formula IIb has a solubility in water of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in water that is within a range defined by any two of the preceding values.

In some embodiments, the compound of Formula IIa or Formula IIb has a solubility in an organic solvent (e.g., acetone, ethanol, chloroform, toluene) of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in an organic solvent that is within a range defined by any two of the preceding values.

In some embodiments, when the composition of Formula Ia or Formula IIb is reacted with parahydrogen, the chemical yield (e.g., chemical yield of a compound of Formula IIa or Formula IIb) is at least about 30%, 35%, 40%, 45%, 50%, 55%, 60%, 65%, 70%, 75%, 80%, 85%, 90%, 95%, or more, at most about 95%, 90%, 85%, 80%, 75%, 70%, 65%, 60%, 55%, 50%, 45%, 40%, 35%, 30%, or less, or within a range defined by any two of the preceding values. For instance, in some embodiments, when the composition of Formula Ia or Formula Ib is reacted with parahydrogen, the chemical yield is between 30% and 35%, between 30% and 40%, between 30% and 45%, between 30% and 50%, between 30% and 55%, between 30% and 60%, between 30% and 65%, between 30% and 70%, between 30% and 75%, between 30% and 80%, between 30% and 85%, between 30% and 90%, between 30% and 95%, between 35% and 40%, between 35% and 45%, between 35% and 50%, between 35% and 55%, between 35% and 60%, between 35% and 65%, between 35% and 70%, between 35% and 75%, between 35% and 80%, between 35% and 85%, between 35% and 90%, between 35% and 95%, between 40% and 45%, between 40% and 50%, between 40% and 55%, between 40% and 60%, between 40% and 65%, between 40% and 70%, between 40% and 75%, between 40% and 80%, between 40% and 85%, between 40% and 90%, between 40% and 95%, between 45% and 50%, between 45% and 55%, between 45% and 60%, between 45% and 65%, between 45% and 70%, between 45% and 75%, between 45% and 80%, between 45% and 85%, between 45% and 90%, between 45% and 95%, between 50% and 55%, between 50% and 60%, between 50% and 65%, between 50% and 70%, between 50% and 75%, between 50% and 80%, between 50% and 85%, between 50% and 90%, between 50% and 95%, between 55% and 60%, between 55% and 65%, between 55% and 70%, between 55% and 75%, between 55% and 80%, between 55% and 85%, between 55% and 90%, between 55% and 95%, between 60% and 65%, between 60% and 70%, between 60% and 75%, between 60% and 80%, between 60% and 85%, between 60% and 90%, between 60% and 95%, between 65% and 70%, between 65% and 75%, between 65% and 80%, between 65% and 85%, between 65% and 90%, between 65% and 95%, between 70% and 75%, between 70% and 80%, between 70% and 85%, between 70% and 90%, between 70% and 95%, between 75% and 80%, between 75% and 85%, between 75% and 90%, between 75% and 95%, between 80% and 85%, between 80% and 90%, between 80% and 95%, between 85% and 90%, between 85% and 95%, or between 90% and 95%.

### Cleaved Precursors of Formula IIIa and Formula IIIb

In some embodiments, a compound of Formula IIa is cleaved (e.g., hydrolyzed), as described herein. In some embodiments, a compound of Formula IIa is cleaved (e.g., hydrolyzed), as described herein, to provide a sidearm compound and a corresponding biorelevant imaging agent. In some embodiments, cleavage of a compound of Formula IIa yields a compound of Formula IIIa and a corresponding biorelevant imaging agent, as described herein. Formula IIIa encompasses the following structure:

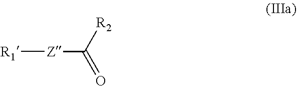

and includes tautomers thereof, deuterated derivatives of those compounds and their tautomers, pharmaceutically acceptable salts thereof, and ¹³C or ¹⁵N enriched derivatives at one or more sites. In some embodiments, Z″ of Formula IIIa is: (i) a parahydrogenated carbon-carbon single bond (—CH*—CH*—) which is substituted to include ¹H (proton), ²H (deuterium), or a combination thereof, or (ii) a parahydrogenated carbon-carbon double bond (—CH*═CH*—) which is substituted to include ¹H (proton), ²H (deuterium), or a combination thereof. In some embodiments, R₁′ of Formula IIIa comprises a PHIP transfer moiety, as described herein. In some embodiments, R₂ of Formula IIIa comprises an optionally substituted hydrocarbon, alkoxy group, primary amine, secondary amine, or tertiary amine, as described herein. In Formula IIIa, all of moieties R₁—Z″—(C═O)—R₂ may be collectively referred to as a cleaved sidearm or a hydrolyzed sidearm.

In some embodiments, a compound of Formula IIb is cleaved (e.g., hydrolyzed), as described herein. In some embodiments, a compound of Formula IIb is cleaved (e.g., hydrolyzed), as described herein, to provide a sidearm compound and a corresponding biorelevant imaging agent. In some embodiments, cleavage of a compound of Formula IIb yields a compound of Formula IIIb and a corresponding biorelevant imaging agent, as described herein. Formula IIIb encompasses the following structure:

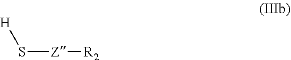

and includes tautomers thereof, deuterated derivatives of those compounds and their tautomers, pharmaceutically acceptable salts thereof, and ¹³C enriched derivatives at one or more sites. In some embodiments, Z″ of Formula IIIb denotes a parahydrogenated ethenyl (—CH*═CH*—) group, an optionally substituted parahydrogenated prop-2-enyl (—C—CH*—CH*—) group, an optionally substituted parahydrogenated ethanyl (—CH*—CH*—) group, an optionally substituted parahydrogenated propanyl (—C—CH*—CH*—) group, or an optionally substituted parahydrogenated butanyl (—C—C—CH*═CH*—) group. R₂ of Formula IIIb comprises an optionally substituted hydrocarbon group, alkyl group, cyclic alkyl group, aryl group, carboxyl group, keto group, or alkoxy group, as described herein. In Formula IIIb, all of moieties H—S—Z′—R₂ may be collectively referred to as a cleaved sidearm or a hydrolyzed sidearm.

In some embodiments, the compound of Formula IIIa or Formula IIIb has a solubility in water of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in water that is within a range defined by any two of the preceding values.

In some embodiments, the compound of Formula IIIa or Formula IIIb has a solubility in an organic solvent (e.g., acetone, ethanol, chloroform, toluene) of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in an organic solvent that is within a range defined by any two of the preceding values.

### Sidearms of Formula IVa

In some embodiments, biorelevant imaging agents and sidearms, such as the sidearm compound of Formula IVa, are conjugated to form precursor compounds, such as the compound of Formula Ia, as described herein. Formula IVa encompasses the following structure:

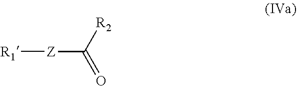

and includes tautomers thereof, deuterated derivatives of those compounds and their tautomers, pharmaceutically acceptable salts thereof, and ¹³C or ¹⁵N enriched derivatives at one or more sites. In some embodiments, Z of Formula IVa describes: (i) a carbon-carbon double bond (—C═C—) which is substituted to include ¹H (proton), ²H (deuterium), or a combination thereof (e.g., —C′H═C¹H—, —C¹H═C²H—, —C′H═C²H—) or (ii) a carbon-carbon triple bond (—C≡C—). In some embodiments, R₁ of Formula IVa comprises a parahydrogen induced polarization (PHIP) transfer moiety, as described herein. In some embodiments, R₂ of Formula IVa comprises a solubilizing moiety, as described herein. In some embodiments, R₂ of Formula IVa comprises an optionally substituted hydrocarbon, alkoxy group, primary amine, secondary amine, or tertiary amine. In some embodiments, conjugation of a compound of Formula IVa with a biorelevant imaging agent yields a compound of Formula Ia, as described herein.

### PHIP Transfer Moieties

In some embodiments, compositions of the present disclosure comprise a PHIP transfer moiety. In some embodiments, compositions of the present disclosure comprise a PHIP transfer moiety between the Z, Z′, or Z″ moiety and the sulfur atom of Formula Ib, Formula IIb, or Formula IIIb. In some embodiments, a PHIP transfer moiety described herein comprises a chemical moiety configured to permit or enhance polarization transfer from one or more parahydrogenated protons H* (e.g., H* in a sidearm) to one or more non-hydrogen nuclear spins of a biorelevant imaging agent (such as one or more ¹³C or ¹⁵N atoms of a biorelevant imaging agent, as described herein). In some embodiments, the PHIP transfer moiety permits or enhances polarization transfer from the parahydrogen protons H* in the sidearm of the compound of Formula IIa or Formula IIb to the non-hydrogen nuclear spins of the corresponding biorelevant imaging agent of the compound of Formula IIa or Formula IIb. In some embodiments, the PHIP transfer moiety permits or enhances polarization transfer from the parahydrogen protons H* in the sidearm of the compound of Formula IIa or Formula IIb to the non-hydrogen nuclear spins of the corresponding biorelevant imaging agent of the compound of Formula IIa or Formula IIb, following the parahydrogenation reaction between parahydrogen and Formula Ia or Formula IIa.

In some embodiments, the PHIP transfer moiety comprises an optionally substituted C1 hydrocarbon or an optionally substituted C2 hydrocarbon.

In some embodiments, the PHIP transfer moiety comprises a chemical moiety of the form *CR₄R₅, *CR₄Y, *C═Y, or any deuterated version thereof. In some embodiments, *C is a ¹²C or ¹³C carbon isotope. In some embodiments, R₄ and R₅ are each independently selected from: ¹H, ²H, ³H, a linear, branched, or cyclic C1-C10 alkyl hydrocarbon, a C6 aryl, a benzyl, a phenyl, a heteroaryl, and a haloalkyl group. In some embodiments, Y is selected from: a spin-1/2 atom, and a spin-1/2 atom covalently bonded to one or more chemical moiety chosen from: a linear, branched, or cyclic C1-C10 alkyl hydrocarbon, a C6 aryl, benzyl, phenyl, heteroaryl, halogen or haloalkyl group, or a heteroatom such as N, O, S, optionally substituted with a linear, branched, or cyclic C1-C10 alkyl hydrocarbon, a C6 aryl, benzyl, phenyl, heteroaryl, halogen or haloalkyl group. In some embodiments, the spin-1/2 atom is selected from: ¹H, ¹³C, ¹⁵N, ¹⁹F, and ³¹P. In some embodiments, the ¹⁵N can be substituted with a nitro group, amine group, amide group, or imine group. In some embodiments, the 31p can be substituted with one or more keto groups, one or more nitro groups, one or more amine groups, one or more amide groups, or one or more imine groups.

In some embodiments, the PHIP transfer moiety comprises a chemical moiety of the form *CR₆R₇—*CR₈R₉, or any deuterated version thereof. In some embodiments, *C is a ¹²C or ¹³C carbon isotope. In some embodiments, R₆, R₇, R₈, and R₉ are each independently selected from: 1H, 2H, 3H, a linear, branched, or cyclic C1-C10 alkyl hydrocarbon, a C6 aryl, a benzyl, a phenyl, a heteroaryl, and a haloalkyl group.

In some embodiments, the PHIP transfer moiety comprises a chemical moiety of the form *CH₂, *CH₂—*CH₂, *CHY, *C═Y, or any deuterated version thereof. In some embodiments, *C is a ¹²C or ¹³C carbon isotope. In some embodiments, Y is selected from: a spin-1/2 atom, and a spin-1/2 atom covalently bonded to one or more chemical moiety chosen from: a linear, branched, or cyclic C1-C10 alkyl hydrocarbon, a C6 aryl, benzyl, phenyl, heteroaryl, halogen or haloalkyl group, or a heteroatom such as N, O, S, optionally substituted with a linear, branched, or cyclic C1-C10 alkyl hydrocarbon, a C6 aryl, benzyl, phenyl, heteroaryl, halogen or haloalkyl group. In some embodiments, the spin-1/2 atom is selected from: 1H, ¹³C, ¹⁵N, ¹⁹F, and ³¹P

In some embodiments, the compositions described herein comprise a first J-coupling J₁₂ between a spin-1/2 atom described herein and a non-hydrogen nuclear spin described herein. In some embodiments, the compositions described herein comprise a second J-coupling J₁₃ between the spin-1/2 atom described herein and a parahydrogen protons H* described herein. In some embodiments, the compositions described herein comprise a third J-coupling J₂₃ between the non-hydrogen nuclear spin described herein and the parahydrogen protons H* described herein. In some embodiments, J₁₂ and/or J₁₃ is greater than J₂₃. In such cases, the PHIP transfer moiety may permit or enhance polarization transfer.

In some embodiments, the PHIP transfer moiety induces a J-coupling between one or both of the *H nuclear spins with the non-hydrogen nuclear spins of at least about 0.1 Hz, 0.2 Hz, 0.3 Hz, 0.4 Hz, 0.5 Hz, 0.6 Hz, 0.7 Hz, 0.8 Hz, 0.9 Hz, 1 Hz, 2 Hz, 3 Hz, 4 Hz, 5 Hz, 6 Hz, 7 Hz, 8 Hz, 9 Hz, 10 Hz, or more, at most about 10 Hz, 9 Hz, 8 Hz, 7 Hz, 6 Hz, 5 Hz, 4 Hz, 3 Hz, 2 Hz, 1 Hz, 0.9 Hz, 0.8 Hz, 0.7 Hz, 0.6 Hz, 0.5 Hz, 0.4 Hz, 0.3 Hz, 0.2 Hz, 0.1 Hz, or less, or a J-coupling with the non-hydrogen nuclear spins that is within a range defined by any two of the preceding values. For instance, in some embodiments, the J-coupling is between 1 Hz and 2 Hz, between 1 Hz and 3 Hz, between 1 Hz and 4 Hz, between 1 Hz and 5 Hz, between 1 Hz and 6 Hz, between 1 Hz and 7 Hz, between 1 Hz and 8 Hz, between 1 Hz and 9 Hz, between 1 Hz and 10 Hz, between 2 Hz and 3 Hz, between 2 Hz and 4 Hz, between 2 Hz and 5 Hz, between 2 Hz and 6 Hz, between 2 Hz and 7 Hz, between 2 Hz and 8 Hz, between 2 Hz and 9 Hz, between 2 Hz and 10 Hz, between 3 Hz and 4 Hz, between 3 Hz and 5 Hz, between 3 Hz and 6 Hz, between 3 Hz and 7 Hz, between 3 Hz and 8 Hz, between 3 Hz and 9 Hz, between 3 Hz and 10 Hz, between 4 Hz and 5 Hz, between 4 Hz and 6 Hz, between 4 Hz and 7 Hz, between 4 Hz and 8 Hz, between 4 Hz and 9 Hz, between 4 Hz and 10 Hz, between 5 Hz and 6 Hz, between 5 Hz and 7 Hz, between 5 Hz and 8 Hz, between 5 Hz and 9 Hz, between 5 Hz and 10 Hz, between 6 Hz and 7 Hz, between 6 Hz and 8 Hz, between 6 Hz and 9 Hz, between 6 Hz and 10 Hz, between 7 Hz and 8 Hz, between 7 Hz and 9 Hz, between 7 Hz and 10 Hz, between 8 Hz and 9 Hz, between 8 Hz and 10 Hz, or between 9 Hz and 10 Hz.

### R₂ Groups

In some embodiments, an R₂ group described herein comprises an optionally substituted hydrocarbon, alkoxy group, primary amine, secondary amine, or tertiary amine. In some embodiments, an R₂ group described herein comprises an optionally substituted hydrocarbon, alkoxy group, primary amine, secondary amine, or tertiary amine that functions as a solubilizing moiety. In some embodiments, an R₂ group described herein comprises a solubilizing moiety. In some embodiments, the solubilizing moiety comprises any chemical moiety configured to permit or enhance the solubility of a compound, such as any of the compounds of Formula Ia, Ib, IIa, IIb, IIIa, IIIb and/or IVa in a solution in which the parahydrogenation reaction or the cleavage (e.g., hydrolysis) reaction takes place. In some embodiments, the enhancement of the solubility is measured with respect to a variant of the compound of Formula Ia, Ib, IIa, IIb, IIIa, IIIb and/or IVa that utilizes one or more protons in place of the R₂ group. In some embodiments, the enhancement of the solubility is measured with respect to a variant of the compound of Formula Ia, Ib, IIa, IIb, IIIa, IIIb and/or IVa that utilizes a methyl group as the R₂ group.

In some embodiments, the solubilizing moiety comprises a hydrophobic moiety or an organophilic moiety. In some embodiments, the solubilizing moiety comprises an organic solubilizing moiety. For example, in some embodiments, the solubilizing moiety comprises a hydrophobic moiety, an organophilic moiety, or an organic solubilizing moiety. In some embodiments, the solubilizing moiety comprises a hydrophilic moiety or an organophobic moiety.

In some embodiments, the R₂ group comprises, or is selected from: a methyl group, an ethyl group, a propyl group, an isopropyl group, an n-butyl group, an s-butyl group, a t-butyl group, an isobutyl group, a hydroxy group, a methyl alcohol group, an ethyl alcohol group, an n-propanol group, an isopropyl alcohol group a propionic alcohol group, an n-butyl alcohol group, an s-butyl alcohol group, a t-butyl alcohol group, an isobutyl alcohol group, a methoxy group, an ethoxy group, a propoxy group, an isopropxy, a propionic group, a butoxy group, a t-butoxy group, a s-butoxy group, an ester group, a phenyl group, a substituted phenyl group, a primary amine group, a secondary amine group, a tertiary amine group, a primary amide group, a secondary amide group, and a tertiary amide group. In some embodiments, the substituted phenyl group is selected from fluorobenzene, chlorobenzene, bromobenzene, iodobenzene, toluene, cumene, ethylbenzene, styrene, ortho-xylene, meta-xylene, para-xylene, phenol, benzoic acid, benzaldehyde, acetophenone, methyl benzoate, anisole, aniline, nitrobenzene, benzonitrile, benzamide, benzenesulfonic acid, naphthalene, and anthracene.

### R₃ Groups

In some embodiments, an R₃ group described herein comprises a biorelevant imaging agent. In some embodiments, the biorelevant imaging agent has the formula R₄C(═O)X—. In some embodiments, R₄ is chosen from a linear, branched, or cyclic C1-C10 alkyl group, in which one or more C atoms are optionally substituted with CO, COOH, CH₂COOH, CONH₂, an OH, an amino (NR′R″), one or more halogen atoms, one or more halo-alkyl groups, or one or more carbocycles, wherein the carbocycle is optionally substituted with one or more aliphatic or aromatic ring, which is optionally substituted by one or more functional groups. In some embodiments, X is chosen from NR″″′ and O. In some embodiments, R′, R″, and R′″ are each independently selected from 1H, 2H, 3H, and an amino protecting group, optionally selected from trifluoroacetyl, acetyl, benzoyl, carbobenzoxy, tert-butyl carbonate and benzyl. In some embodiments, the R₃ group comprises any biorelevant imaging agent described herein.

In some embodiments, an R₃ group described herein comprises an acyl derivative of a biorelevant imaging agent. In some embodiments, the biorelevant imaging agent has the formula R₉C(═O)O—. In some embodiments, R₉ is chosen from a linear, branched, or cyclic C1-C10 alkyl group, in which one or more C atoms are optionally substituted with CO, COOH, CH₂COOH, CONH₂, an OH, an amino (NR′R″), one or more halogen atoms, one or more halo-alkyl groups, or one or more carbocycles, wherein the carbocycle is optionally substituted with one or more aliphatic or aromatic ring, which is optionally substituted by one or more functional groups. In some embodiments, R′ and R″ are each independently selected from 1H, 2H, 3H, and an amino protecting group, optionally selected from trifluoroacetyl, acetyl, benzoyl, carbobenzoxy, tert-butyl carbonate and benzyl. In some embodiments, the R₃ group comprises an acyl derivative of any biorelevant imaging agent described herein.

In some embodiments, the R₃ group comprises at least one non-hydrogen nuclear spin. In some embodiments, the non-hydrogen nuclear comprises at least one spin-1/2 atom. In some embodiments, the non-hydrogen nuclear spin comprises ¹³C or ¹⁵N. In some embodiments, the R₃ group is at least partially isotopically labeled with the non-hydrogen nuclear spin. In some embodiments, the R₃ group is at least partially enriched with the non-hydrogen nuclear spin when compared to an analog of the R₃ group that features the non-hydrogen nuclear spin at its natural abundance. In some embodiments, the R₃ group is enriched to feature the non-hydrogen nuclear spin at an abundance of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more, at most about 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less, or an abundance that is within a range defined by any two of the preceding values.

In some embodiments, the non-hydrogen nuclear spin replaces an NMR-inactive (i.e., spin-0) nucleus (e.g., ¹²C or a quadrupolar (i.e., spin >1/2) nucleus (e.g., ¹⁴N) of the analog of the R₃ group that features the non-hydrogen nuclear spin at its natural abundance, as described herein. In some embodiments, the non-hydrogen nuclear spin is located no more than about 1 or 2 chemical bonds from the carbonyl (C═O) carbon in the R₃ group.

### Parahydrogenation

In some embodiments, the non-hydrogen nuclear spin replaces an NMR-inactive (i.e., spin-0) nucleus (e.g., ¹²C or a quadrupolar (i.e., spin >1/2) nucleus (e.g., 14N) of the analog of the R₃ group that features the non-hydrogen nuclear spin at its natural abundance, as described herein. In some embodiments, the non-hydrogen nuclear spin is located no more than about 1 or 2 chemical bonds from the carbonyl (C═O) carbon in the R₃ group.

Consistent with disclosed embodiments, a precursor to the biorelevant imaging agent (such as a compound of Formula Ia or Formula Ib, as described herein) can be parahydrogenated by combining the precursor, parahydrogen, and a hydrogenation catalyst. The disclosed embodiments are not limited to a particular method of generating a parahydrogenated precursor. In some embodiments, the precursor is added to a mixture containing parahydrogen. In some embodiments, parahydrogen gas is added to a solution containing the precursor (e.g., the parahydrogen gas can be bubbled into such a solution). In hydrogenating the precursor, the parahydrogen can create Iz1Iz2 order, preferential population of the lower energy state between |1>|>, |>|1> or singlet spin order on two hydrogens spins in the precursor.

The precursor can have an unsaturated bond (such as an unsaturated carbon-carbon double bond or an unsaturated carbon-carbon triple bond) that can be hydrogenated by the parahydrogen gas. Following combination of the precursor and the parahydrogen, at least about 10%, 20%, 30%, 40%, 50%, 60%, 70%, 80%, 90%, or more of the precursor, at most about 90%, 80%, 70%, 60%, 50%, 40%, 30%, 20%, 10%, or less of the precursor, or a percentage of the precursor that is within a range defined by any two of the preceding values may be hydrogenated.

In some embodiments, the parahydrogenated precursor has a population difference in the parahydrogenated proton spin states of at least about 1%, 2%, 3%, 4%, 5%, 6%, 7%, 8%, 9%, 10%, 15%, 20%, 25%, 30%, 40%, 45%, 50%, or more, at most about 50%, 45%, 40%, 35%, 30%, 25%, 20%, 15%, 10%, 9%, 8%, 7%, 6%, 5%, 4%, 3%, 2%, 1%, or less, or a population difference that is within a range defined by any two of the preceding values. For instance, in some embodiments, the population difference is between 10% and 15%, between 10% and 20%, between 10% and 25%, between 10% and 30%, between 10% and 35%, between 10% and 40%, between 10% and 45%, between 10% and 50%, between 15% and 20%, between 15% and 25%, between 15% and 30%, between 15% and 35%, between 15% and 40%, between 15% and 45%, between 15% and 50%, between 20% and 25%, between 20% and 30%, between 20% and 35%, between 20% and 40%, between 20% and 45%, between 20% and 50%, between 25% and 30%, between 25% and 35%, between 25% and 40%, between 25% and 45%, between 25% and 50%, between 30% and 35%, between 30% and 40%, between 30% and 45%, between 30% and 50%, between 35% and 40%, between 35% and 45%, between 35% and 50%, between 40% and 45%, between 40% and 50%, or between 45% and 50%. In some embodiments, the population difference is between spin states which include the parahydrogenated protons as well as other nuclear spins, for example additional protons on the compound. In some embodiments, the parahydrogenated precursor includes a sidearm and the parahydrogenated spins can be located on the sidearm.

In some embodiments, the concentration of the hydrogenation catalyst during hydrogenation is at least about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, or more, at most about 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, 0.9 mM, 0.8 mM, 0.7 mM, 0.6 mM, 0.5 mM, 0.4 mM, 0.3 mM, 0.2 mM, 0.1 mM, or less, or within a range defined by any two of the preceding values.

The disclosed embodiments can include methods implemented by the disclosed systems for generating a hyperpolarized biorelevant imaging agent. The disclosed methods can include mixing (e.g., by a mixing mechanism) a solution which includes a precursor to the biorelevant imaging agent and a hydrogenation catalyst. A mixing mechanism may be a device for introducing, holding, and facilitating a blend, mixture, or solution of two or more materials. In some embodiments, the mixing mechanism is disposed in a chamber, and the mixing occurs inside the chamber. In some embodiments, the solution is mixed at a location away from the chamber. The solution may be at least about 1 milliliter (ml), 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, or more in volume, at most about 100 ml, 90 ml, 80 ml, 70 ml, 60 ml, 50 ml, 40 ml, 30 ml, 20 ml, 10 ml, 9 ml, 8 ml, 7 ml, 6 ml, 5 ml, 4 ml, 3 ml, 2 ml, 1 ml, or less in volume, or within a volume range defined by any two of the preceding values.

In some embodiments, the mixing mechanism is a gas-liquid exchange mechanism. For example, the gas-liquid exchange mechanism may be a bubbler or a diffusion system. In some embodiments, the mixing mechanism comprises membranes adapted to permit diffusion of molecular hydrogen. In some embodiments the mixing can be performed using a spray chamber, where the solution is sprayed into a chamber filled with pressurized parahydrogen.

In some embodiments, the catalyst is a molecule, complex or particle system that catalyzes hydrogenation. In some embodiment, the catalyst comprises a homogeneous metal catalyst such as a rhodium complex or a ruthenium complex. The rhodium complex can be used for coordination and activation of precursor molecules and parahydrogen. In some embodiments, a heterogeneous metal catalyst is connected to a nanoparticle.

Various embodiments of the present disclosure describe introducing a solution which includes a precursor to the biorelevant imaging agent and a hydrogenation catalyst into a chamber configured to hold the solution during polarization transfer. In some embodiments, the solution is mixed in the chamber. In some embodiments, the solution is hydrogenated in the chamber. In some embodiments, the chamber is within a magnetic shield (e.g., a mu metal shield). The magnetic shield can reduce the effect of the Earth's magnetic field (or other extraneous magnetic fields), permitting modulation of the amplitude of a low-level magnetic field applied to the solution. Accordingly, placing the solution within the chamber can include placing the solution within the magnetic shield.

As described herein, in some embodiments parahydrogenation occurs prior to polarization transfer (e.g., prior to the modulation of the amplitude the magnetic field applied to the solution, or the like). In some embodiments, parahydrogenation occurs during polarization transfer. For example, parahydrogen can be combined with (e.g., flowed or bubbled through the solution) the solution during modulation of the amplitude of the magnetic field.

In some embodiments, the parahydrogen gas is combined with the solution in a hydrogenation chamber at pressure. The pressure can be at least about 10 bar, 15 bar, 20 bar, 30 bar, 50 bar, or more, at most about 50 bar, 30 bar, 20 bar, 15 bar, 10 bar or less, or within a range defined by any two of the preceding values. In some embodiments, the parahydrogen is combined with the solution in a metallic chamber capable of withstanding the pressure. The parahydrogen can be combined with the solution for (or the dissolution of the parahydrogen can occur in less than) a time interval. The time interval can be at most about 90 seconds, 60 seconds, 30 seconds, 20 seconds, 10 seconds, 9 seconds, 8 seconds, 7 seconds, 6 seconds, 5 seconds, 4 seconds, 3 seconds, 2 seconds, 1 second, or less, at least about 1 second, 2 seconds, 3 seconds, 4 seconds, 5 seconds, 6 seconds, 7 seconds, 8 seconds, 9 seconds, 10 seconds, 20 seconds, 30 seconds, 60 seconds, 90 seconds, or more, or within a range defined by any two of the preceding values. In some embodiments, the hydrogenation is carried out or occurs within the time interval.

### Polarization Transfer Using Radiofrequency Waveforms

In some embodiments, the concentration of the precursor or target molecule in the solution prior to polarization transfer is at least about 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, 1,000 mM, or more, at most about 1,000 mM, 900 mM, 800 mM, 700 mM, 600 mM, 500 mM, 400 mM, 300 mM, 200 mM, 100 mM, 90 mM, 80 mM, 70 mM, 60 mM, 50 mM, 40 mM, 30 mM 20 mM, 10 mM, or less, or within a range defined by any two of the preceding values. The volume of the solution can be at least about 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, 200 ml, 300 ml, 400 ml, 500 ml, 600 ml, 700 ml, 800 ml, 900 ml, 1000 ml, 2000 ml, or more, at most about 2000 ml, 1000 ml, 900 ml, 800 ml, 700 ml, 600 ml, 500 ml, 400 ml, 300 ml, 200 ml, 100 ml, 90 ml, 80 ml, 70 ml, 60 ml, 50 ml, 40 ml, 30 ml, 20 ml, 10 ml, 9 ml, 8 ml, 7 ml, 6 ml, 5 ml, 4 ml, 3 ml, 2 ml, 1 ml, or less, or within a range defined by any two of the preceding values.

Various embodiments of the present disclosure describe applying a polarization transferring magnetic perturbation aimed to generate a magnetic field around the solution (e.g., around a solution containing Formula IIa or Formula IIb described herein). In some embodiments, the magnetic field has a strength of at least about 0.1 gauss (G), 0.2 G, 0.3 G, 0.4 G, 0.5 G, 0.6 G, 0.7 G, 0.8 G, 0.9 G, 1 G, 2 G, 3 G, 4 G, 5 G, 6 G, 7 G, 8 G, 9 G, 10 G, 20 G, 30 G, 40 G, 50 G, 60 G, 70 G, 80 G, 90 G, 100 G, 200 G, 300 G, 400 G, 500 G, 600 G, 700 G, 800 G, 900 G, 1,000 G, 2,000 G, 3,000 G, 4,000 G, 5,000 G, 6,000 G, 7,000 G, 8,000 G, 9,000 G, 10,000 G, 20,000 G, 30,000 G, 40,000 G, 50,000 G, 60,000 G, 70,000 G, 80,000 G, 90,000 G, 100,000 G, 200,000 G, or more, at most about 200,000 G, 100,000 G, 90,000 G, 80,000 G, 70,000 G, 60,000 G, 50,000 G, 40,000 G, 30,000 G, 20,000 G, 10,000 G, 9,000 G, 8,000 G, 7,000 G, 6,000 G, 5,000 G, 4,000 G, 3,000 G, 2,000 G, 1,000 G, 900 G, 800 G, 700 G, 600 G, 500 G. 400 G, 300 G. 200 G, 100 G, 90 G, 80 G, 70 G, 60 G, 50 G, 40 G, 30 G, 20 G, 10 G, 9 G, 8 G, 7 G, 6 G, 5 G, 4 G, 3 G, 2 G, 1 G, 0.9 G, 0.8 G, 0.7 G, 0.6 G, 0.5 G, 0.4 G, 0.3 G, 0.2 G, 0.1 G, or less, or within a range defined by any two of the preceding values. In some embodiments, the magnetic field has a strength of 0.1 G to 200,000 G around the solution. The magnetic perturbation can be produced by an electro-magnet or a permanent magnet. The magnetic field can be applied to the sample in pulses or in a continuous wave (CW). The magnetic perturbation can be static or time varying.

A signal generator can be configured to generate one or more radiofrequency (RF) waveforms that can be applied to the sample to transfer polarization. The signal generator can include one more computing unit, processors, controllers, associate memories, PCs, computers services, or any devices capable of carrying computational operations using inputs and producing outputs. In some embodiments, RF coils may radiate, or ‘apply’ the pulse sequences, including the first RF waveform. In some embodiments, the RF coils may have one or more channels. Channels may be pathways for RF signals. There may be provided at least one channel for each different type of NMR spectroscopy. In some embodiments, there is at least one channel for 1H and at least one channel for any of 2H, ¹³C, ¹⁵N, ¹⁹F, and ³¹P For example, a first RF waveform can be applied to a ¹H channel of the one or more radiofrequency coils (RF coils) disposed around the sample. In some embodiments, a second RF waveform is applied to a ¹³C channel of the RF coils. In some embodiments, the RF waveforms on the ¹H channel and ¹³C channel are configured to apply a polarization transfer sequence, such as PH-INEPT, Goldman's sequence, S2M, S2hM, SLIC, ADAPT or ESOTERIC.

In some embodiments, the RF waveforms is configured to support polarization transfer, even in the presence of a large proton full width half maximum (FWHM). Such RF waveforms can include a pulse sequence, which can include tens to hundreds of RF pulses. The sequence can be configured such that the pulses protect against the detrimental effects of magnetic field inhomogeneities on polarization transfer.

In some embodiments, a pulse sequence for polarization is configured to transfer the spin order from non-equivalent two 1H hydrogenated spins, e.g., when the chemical shift difference is larger than the J-coupling between them. ESOTHERIC, for example, may be a pulse sequence suited for polarization transfer in this regime.

In some embodiments, the pulse sequence is configured to transfer the spin order from equivalent 1H hydrogen spins, e.g., when the chemical shift difference is smaller than the J-coupling between them. Such pulse sequences may be used in magnetic fields having a strength of at least about 0.01 millitesla (mT), 0.02 mT, 0.03 mT, 0.04 mT, 0.05 mT, 0.06 mT, 0.07 mT, 0.08 mT, 0.09 mT, 0.1 mT, 0.2 mT, 0.3 mT, 0.4 mT, 0.5 mT, 0.6 mT, 0.7 mT, 0.8 mT, 0.9 mT, 1 mT, 2 mT, 3 mT, 4 mT, 5 mT, 6 mT, 7 mT, 8 mT, 9 mT, 10 mT, 20 mT, 30 mT, 40 mT, 50 mT, 60 mT, 70 mT, 80 mT, 90 mT, 100 mT, 200 mT, 300 mT, 400 mT, 500 mT, 600 mT, 700 mT, 800 mT, 900 mT, 1,000 mT, 2,000 mT, 3,000 mT, 4,000 mT, 5,000 mT, 6,000 mT, or more, at most about 6,000 mT, 5,000 mT, 4,000 mT, 3,000 mT, 2,000 mT, 1,000 mT, 900 mT, 800 mT, 700 mT, 600 mT, 500 mT, 400 mT, 300 mT, 200 mT, 100 mT, 90 mT, 80 mT, 70 mT, 60 mT, 50 mT, 40 mT, 30 mT, 20 mT, 10 mT, 9 mT, 8 mT, 7 mT, 6 mT, 5 mT, 4 mT, 3 mT, 2 mT, 1 mT, 0.9 mT, 0.8 mT, 0.7 mT, 0.6 mT, 0.5 mT, 0.4 mT, 0.3 mT, 0.2 mT, 0.1 mT, 0.09 mT, 0.08 mT, 0.07 mT, 0.06 mT, 0.05 mT, 0.04 mT, 0.03 mT, 0.02 mT, 0.01 mT, or less, or within a range defined by any two of the preceding values. An example of such a sequence may be Goldman's sequence (M. Goldman, H. Jóhannesson, C. R. Phys. 2005, 6, 575-581, which is incorporated herein by reference as related to pulse sequence configurations to transfer spin order), the singlet to heteronuclear magnetization (S2hM) sequence, or other sequences used in singlet NMR (e.g., ADAPT, SLIC, etc.).

In some embodiments, a magnetic shield is configured to maintain a magnetic field applied to the solution of at least about 0 mG, 0.1 mG, 0.2 mG, 0.3 mG, 0.4 mG, 0.5 mG, 0.6 mG, 0.7 mG, 0.8 mG, 0.9 mG, 1 mG, 2 mG, 3 mG, 4 mG, 5 mG, 6 mG, 7 mG, 8 mG, 9 mG, 10 mG, 20 mG, 30 mG, 40 mG, 50 mG, 60 mG, 70 mG, 80 mG, 90 mG, 100 mG, or more, at most about 100 mG, 90 mG, 80 mG, 70 mG, 60 mG, 50 mG, 40 mG, 30 mG, 20 mG, 10 mG, 9 mG, 8 mG, 7 mG, 6 mG, 5 mG, 4 mG, 3 mG, 2 mG, 1 mG, 0.9 mG, 0.8 mG, 0.7 mG, 0.6 mG, 0.5 mG, 0.4 mG, 0.3 mG, 0.2 mG, 0.1 mG or less, or a magnetic field that is within a range defined by any two of the preceding values. The magnetic shield can maintain the magnetic field strength within the polarization chamber at such amplitudes during application of the polarization waveform to the one or more radiofrequency coils.

Consistent with disclosed embodiments, the RF waveform can be applied to a solution containing a parahydrogenated precursor.

### Transferring Polarization Using Magnetic Field Modulation

In some embodiments, the polarization transfer magnetic perturbation is performed in a magnetic shield (e.g., a mu shield, or the like) to achieve a homogenous, low magnetic field. The magnetic shield enables performance of polarization transfer to ¹³C nuclear spins at microtesla (μT) magnetic fields, below the earth's magnetic field. The low magnetic field can be at least about 0 mG, 0.1 mG, 0.2 mG, 0.3 mG, 0.4 mG, 0.5 mG, 0.6 mG, 0.7 mG, 0.8 mG, 0.9 mG, 1 mG, 2 mG, 3 mG, 4 mG, 5 mG, 6 mG, 7 mG, 8 mG, 9 mG, 10 mG, 20 mG, 30 mG, 40 mG, 50 mG, 60 mG, 70 mG, 80 mG, 90 mG, 100 mG, or more, at most about 100 mG, 90 mG, 80 mG, 70 mG, 60 mG, 50 mG, 40 mG, 30 mG, 20 mG, 10 mG, 9 mG, 8 mG, 7 mG, 6 mG, 5 mG, 4 mG, 3 mG, 2 mG, 1 mG, 0.9 mG, 0.8 mG, 0.7 mG, 0.6 mG, 0.5 mG, 0.4 mG, 0.3 mG, 0.2 mG, 0.1 mG, or less, or within a range defined by any two of the preceding values.

At such fields, the polarization is transferred by utilizing level avoided crossings (LAC) between the proton spins and other spin species of interest, including 2H, ¹³C, ¹⁵N, ¹⁹F, and ³¹P. In some embodiments, the magnetic field can be tuned to a specific magnetic field strength for the LAC, for example as performed in SABRE-SHEATH experiments. In various embodiments, to enable robust polarization transfer in larger-volume samples, the magnetic field strength can be temporally modulated. For example, the magnetic field strength can be swept through the LAC conditions. Alternatively or additionally, the sample can be physically moved inside the magnetic field. Such modulation can relax constraints on magnetic field homogeneity and on magnetic field offsets. Thus, robust polarization transfer can be performed at larger volumes and with greater efficiency. Furthermore, relaxing the constraints on magnetic field homogeneity and on magnetic field offsets can permit using of less complex, precise, or expensive polarization systems.

A lower bound of the magnetic field modulation can at least about −10 μT, −9 μT, −8 μT, −7 μT, −6 μT, −5 μT, −4 μT, −3 μT, −2 μT, −1 μT, −0.9 μT, −0.8 μT, −0.7 μT, −0.6 μT, −0.5 μT, −0.4 μT, −0.3 μT, −0.2 μT, −0.1 μT, or more, at most about −0.1 μT, −0.2 μT, −0.3 μT, −0.4 μT, −0.5 μT, −0.6 μT, −0.7 μT, −0.8 μT, −0.9 μT, −1 μT, −2 μT, −3 μT, −4 μT, −5 μT, −6 μT, −7 μT, −8 μT, −9 μT, −10 μT, or less, or within a range defined by any two of the preceding values. An upper bound of the modulation can be at least about 0.1 μT, 0.2 μT, 0.3 μT, 0.4 μT, 0.5 μT, 0.6 μT, 0.7 μT, 0.8 μT, 0.9 μT, 1 μT, 2 μT, 3 μT, 4 μT, 5 μT, 6 μT, 7 μT, 8 μT, 9 μT, 10μT, or more, at most about 10 μT, 9 μT, 8 μT, 7 μT, 6 μT, 5 μT, 4 μT, 3 μT, 2 μT, 1 μT, 0.9 μT, 0.8 μT, 0.7 μT, 0.6 μT, 0.5 μT, 0.4 μT, 0.3 μT, 0.2 μT, 0.1 μT, or less, or within a range defined by any two of the preceding values.

The magnetic field can have such an amplitude over a volume of at least about 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, 200 ml, 300 ml, 400 ml, 500 ml, 600 ml, 700 ml, 800 ml, 900 ml, 1,000 ml, 2,000 ml, or more, at most about 2,000 ml, 1,000 ml, 900 ml, 800 ml, 700 ml, 600 ml, 500 ml, 400 ml, 300 ml, 200 ml, 100 ml, 90 ml, 80 ml, 70 ml, 60 ml, 50 ml, 40 ml, 30 ml, 20 ml, 10 ml, 9 ml, 8 ml, 7 ml, 6 ml, 5 ml, 4 ml, 3 ml, 2 ml, 1 ml, or less, or a volume that is within a range defined by any two of the preceding values. The modulation can be performed over a duration. The duration can be at least about 100 milliseconds (ms), 200 ms, 300 ms, 400 ms, 500 ms, 600 ms, 700 ms, 800 ms, 900 ms, 1,000 ms, 2,000 ms, 3,000 ms, 4,000 ms, 5,000 ms, 6,000 ms, 7,000 ms, 8,000 ms, 9,000 ms, 10,000 ms, 20,000 ms, 30,000 ms, 40,000 ms, 50,000 ms, or more, at most about 50,000 ms, 40,000 ms, 30,000 ms, 20,000 ms, 10,000 ms, 9,000 ms, 8,000 ms, 7,000 ms, 6,000 ms, 5,000 ms, 4,000 ms, 3,000 ms, 2,000 ms, 1,000 ms, 900 ms, 800 ms, 700 ms, 600 ms, 500 ms, 400 ms, 300 ms, 200 ms, 100 ms, or less, or within a range defined by any two of the preceding values.

Accordingly, the rate of change of the amplitude of the magnetic field can be at least about 0.01 μT per second, 0.02 μT per second, 0.03 μT per second, 0.04 μT per second, 0.05 μT per second, 0.06 μT per second, 0.07 μT per second, 0.08 μT per second, 0.09 μT per second, 0.1 μT per second, 0.2 μT per second, 0.03 μT per second, 0.4 μT per second, 0.5 μT per second, 0.6 μT per second, 0.7 μT per second, 0.8 μT per second, 0.9 μT per second, 1 μT per second, or more, at most about 1 μT per second, 0.9 μT per second, 0.8 μT per second, 0.7 μT per second, 0.6 μT per second, 0.5 μT per second, 0.4 μT per second, 0.3 μT per second, 0.2 μT per second, 0.1 μT per second, or less, or within a range defined by any two of the preceding values. The upper bound on the rate of change of the amplitude of the magnetic field may be determined by the capabilities of the equipment used to perform the sweep.

In some embodiments, when the magnetic field is within the upper and lower bounds, disclosed above, the spatial deviation of the magnetic field over the volume during modulation is less than about half (or a quarter, or an eighth, or a tenth) of the amplitude of the magnetic field. For example, when the magnetic field strength is less than 2 μT (or greater than-2 μT) then the spatial deviation of the magnetic field over the volume during modulation can be less than 1 μT. As an additional example, when the magnetic field strength is less than 10 μT (or greater than-10 μT) then the spatial deviation of the magnetic field over the volume during modulation can be less than 5 μT. The spatial deviation can be measured for example by taking at least about 10, 20, 30, 40, 50, 60, 70, 80, 90, 100, 200, 300, 400, 500, or more spatially randomly sampled or spatially equally distributed measurements of the magnetic field within the volume and calculating the standard deviation of the sampled magnetic field measurements. Such homogeneity can be achieved for example in a large homogeneous magnetic shield by having a large piercing solenoid through the magnetic shield or by using large Helmholtz coils with a large homogeneous region for producing the magnetic field amplitude modulation. In some embodiments the modulation is a sweep of the magnetic field. In some embodiments, the magnetic field amplitude modulation includes a diabatic jump, monotonous amplitude variation or combinations thereof.

In some embodiments, following the polarization transfer step, a non-hydrogen nuclear spin of the biorelevant imaging agent (such as a ¹³C or ¹⁵N of the biorelevant imaging agent) has nuclear spin polarization of at least about 1%, 2%, 3%, 4%, 5%, 6%, 7%, 8%, 9%, 10%, 15%, 20%, 25%, 30%, 35%, 40%, 45%, 50%, or more, at most about 50%, 45%, 40%, 35%, 30%, 25%, 20%, 15%, 10%, 9%, 8%, 7%, 6%, 5%, 4%, 3%, 2%, 1% or less, or a polarization that is within a range defined by any two of the preceding values. For example, in some embodiments, following the polarization transfer step, a non-hydrogen nuclear spin of the biorelevant imaging agent has nuclear spin polarization between 10% and 15%, between 10% and 20%, between 10% and 25%, between 10% and 30%, between 10% and 35%, between 10% and 40%, between 10% and 45%, between 10% and 50%, between 15% and 20%, between 15% and 25%, between 15% and 30%, between 15% and 35%, between 15% and 40%, between 15% and 45%, between 15% and 50%, between 20% and 25%, between 20% and 30%, between 20% and 35%, between 20% and 40%, between 20% and 45%, between 20% and 50%, between 25% and 30%, between 25% and 35%, between 25% and 40%, between 25% and 45%, between 25% and 50%, between 30% and 35%, between 30% and 40%, between 30% and 45%, between 30% and 50%, between 35% and 40%, between 35% and 45%, between 35% and 50%, between 40% and 45%, between 40% and 50%, or between 45% and 50%.

In some embodiments this polarization is achieved for a solution volume of at least about 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, 200 ml, 300 ml, 400 ml, 500 ml, or more, at most about 500 ml, 400 ml, 300 ml, 200 ml, 100 ml, 90 ml, 80 ml, 70 ml, 60 ml, 50 ml, 40 ml, 30 ml, 20 ml, 10 ml, 9 ml, 8 ml, 7 ml, 6 ml, 5 ml, 4 ml, 3 ml, 2 ml, 1 ml, or less, or a volume that is within a range defined by any two of the preceding values.

In some embodiments, following polarization transfer a portion of the population difference in parahydrogenated proton spin states has been transferred to polarization of the target (e.g., ¹³C or ¹⁵N) nuclear spin of the biorelevant imaging agent. This portion can be at least about 1%, 2%, 3%, 4%, 5%, 6%, 7%, 8%, 9%, 10%, 15%, 20%, 25%, 30%, 35%, 40%, 45%, 50%, or more, at most about 50%, 45%, 40%, 35%, 30%, 25%, 20%, 15%, 10%, 9%, 8%, 7%, 6%5%, 4%, 3%, 2%, 1% or less, or within a range defined by any two of the preceding values. For example, in some embodiments, this portion is between 10% and 15%, between 10% and 20%, between 10% and 25%, between 10% and 30%, between 10% and 35%, between 10% and 40%, between 10% and 45%, between 10% and 50%, between 15% and 20%, between 15% and 25%, between 15% and 30%, between 15% and 35%, between 15% and 40%, between 15% and 45%, between 15% and 50%, between 20% and 25%, between 20% and 30%, between 20% and 35%, between 20% and 40%, between 20% and 45%, between 20% and 50%, between 25% and 30%, between 25% and 35%, between 25% and 40%, between 25% and 45%, between 25% and 50%, between 30% and 35%, between 30% and 40%, between 30% and 45%, between 30% and 50%, between 35% and 40%, between 35% and 45%, between 35% and 50%, between 40% and 45%, between 40% and 50%, or between 45% and 50%.

In some embodiments, the magnetic field modulation includes a diabatic jump of the magnetic field. The diabatic jump can be performed to a magnetic field where a level avoided crossing including the proton spins and a non-proton spin occur. Given the J-couplings between the nuclear spins in the system, this value can be calculated analytically or identified by plotting the energy levels of the Hamiltonian for different magnetic fields and identifying the LAC. In some embodiments, the duration where the magnetic field amplitude is at the LAC condition is at most about 5 seconds, 4 seconds, 3 seconds, 2 seconds, 1 second, 0.9 seconds, 0.8 seconds, 0.7 seconds, 0.6 seconds, 0.5 seconds, 0.4 seconds, 0.3 seconds, 0.2 seconds, 0.1 seconds, or less, at least about 0.1 seconds, 0.2 seconds, 0.3 seconds, 0.4 seconds, 0.5 seconds, 0.6 seconds, 0.7 seconds, 0.8 seconds, 0.9 seconds, 1 seconds, 2 seconds, 3 seconds, 4 seconds, 5 seconds, or more or within a range defined by any two of the preceding values.

In some embodiments, modulation of the amplitude of the magnetic field includes varying the magnetic field amplitude monotonically (or monotonically over each of a limited number of interval-such as one to ten increasing interval and/or one to ten decreasing intervals). In some embodiments, the modulation of the amplitude of the magnetic field comprises linearly varying the amplitude of the magnetic field. The initial magnetic field amplitude of the sweep, the end magnetic field amplitude and the total duration of the sweep can be optimized for the target molecule. In some embodiments the magnetic field amplitude during the sweep is within a lower bound and an upper bound. The lower bound can be at least about −2 μT, −1 μT, −0.9 μT, −0.8 μT, −0.7 μT, −0.6 μT, −0.5 μT, −0.4 μT, −0.3 μT, −0.2 μT, −0.1 μT, or more, at most about −0.1 μT, −0.2 μT, −0.3 μT, −0.4 μT, −0.5 μT, −0.6 μT, −0.7 μT, −0.8 μT, −0.9 μT, −1 μT, −2 μT, or less, or within a range defined by any two of the preceding values. The upper bound can be at least about 0.1 μT, 0.2 μT, 0.3 μT, 0.4 μT, 0.5 μT, 0.6 μT, 0.7 μT, 0.8 μT, 0.9 μT, 1 μT, 2 μT, or more, at most about 2 μT, 1 μT, 0.9 μT, 0.8 μT, 0.7 μT, 0.6 μT, 0.5 μT, 0.4 μT, 0.3 μT, 0.2 μT, 0.1 μT, or less, or within a range defined by any two of the preceding values. In some embodiments the duration of modulation can be at least about 100 ms, 200 ms, 300 ms, 400 ms, 500 ms, 600 ms, 700 ms, 800 ms, 900 ms, 1,000 ms, 2,000 ms, 3,000 ms, 4,000 ms, 5,000 ms, 6,000 ms, 7,000 ms, 8,000 ms, 9,000 ms, 10,000 ms, or more, at most about 10,000 ms, 9,000 ms, 8,000 ms, 7,000 ms, 6,000 ms, 5,000 ms, 4,000 ms, 3,000 ms, 2,000 ms, 1,000 ms, 900 ms, 800 ms, 700 ms, 600 ms, 500 ms, 400 ms, 300 ms, 200 ms, 100 ms, or less, or within a range defined by any two of the preceding values. In some embodiments, the rate of amplitude change is varied along the amplitude profile. In some embodiments, a constant-adiabaticity sweep is calculated by choosing a certain subset of level avoided crossings of the spin system. In some embodiments, the magnetic amplitude modulation includes a combination of diabatic jumps, monotonous amplitude modulation and rate of change sign reversals. In some embodiments, the precursor may be chosen or designed such that following the hydrogenation and other potential chemical reactions, one of the products is a biorelevant imaging agent usable in hyperpolarized NMR or MRI applications.

### Hydrolysis, Purification and Separation

The present disclosure presents methods and systems for producing a composition (e.g., clinical dose composition) which comprises a hyperpolarized biorelevant imaging agent (or a pharmaceutically acceptable salt thereof) in a solvent. In some embodiments, the biorelevant imaging agent is produced through additional chemical reactions and/or processing steps following hydrogenation and polarization transfer, according to the present disclosure. Such additional chemical reactions and/or processing steps may include, but are not limited to: (i) catalyst filtration and scavenging (e.g., filtration scavenging rhodium atoms and/or iridium atoms); (ii) cleaving the sidearm of the biorelevant imaging agent precursor molecule (e.g., cleavage of the compound of Formula IIa or Formula IIb, as described herein) to form the biorelevant imaging agent and a sidearm (e.g., a compound of Formula IIIa or Formula IIIb described herein), e.g., by hydrolysis with an aqueous sodium hydroxide solution; (iii) washing the solution with an organic solvent and separating any resulting aqueous mixture phase from an organic mixture phase; (iv) evaporative extraction of volatile organics from the aqueous mixture (e.g., using nitrogen gas bubbling); and (v) additional filtration/purification/concentration/finishing steps known in the art.

The volume of the solution which includes the biorelevant imaging agent, (e.g., following cleavage) and/or the concentration of the biorelevant imaging agent produced can depend on the volume of the solution used for polarization transfer and concentration of the precursor in that solution. Exemplary ranges of solution volumes and precursor concentrations are described herein. As further specific examples, at least about 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, or more of solution, at most about 100 ml, 90 ml, 80 ml, 70 ml, 60 ml, 50 ml, 40 ml, 30 ml, 20 ml, 10 ml, 9 ml, 8 ml, 7 ml, 6 ml, 5 ml, 4 ml, 3 ml, 2 ml, 1 ml, or less of solution, or an amount of solution that is within a range defined by any two of the preceding values can be produced. In some embodiments, the solution can include at least about 10 mM, 20 mM, 30 mM, 40 mM, 50 mM, 60 mM, 70 mM, 80 mM, 90 mM, 100 mM, 200 mM, 300 mM, 400 mM, 500 mM, or more of the biorelevant imaging agent, at most about 500 mM, 400 mM, 300 mM, 200 mM, 100 mM, 90 mM, 80 mM, 70 mM, 60 mM, 50 mM, 40 mM, 30 mM, 20 mM, 10 mM, or less of the biorelevant imaging agent, or an amount of the biorelevant imaging agent this is within a range defined by any two of the preceding values.

In some embodiments, the present disclosure describes a multi-step liquid-liquid separation and purification process for producing doses (e.g., clinical doses) of a dosage composition comprising the biorelevant imaging agent (e.g., hyperpolarized biorelevant imaging agent or a pharmaceutically acceptable salt thereof).

In some embodiments, the polarization step is followed by the addition of an aqueous mixture (e.g., water) to the solution comprising the first organic solvent and the biorelevant imaging agent. In some embodiments, the first organic solvent and the aqueous mixture (e.g., water) produce a biphasic solution comprising (i) an organic mixture phase comprising the first organic solvent, and (ii) an aqueous mixture phase comprising the aqueous mixture and the hyperpolarized biorelevant imaging agent or a pharmaceutically acceptable salt thereof. In some embodiments, the first organic solvent and aqueous mixture (e.g., water) produce the biphasic solution, wherein a portion of the organic solvent is retained in the aqueous mixture. In some embodiments, the first organic solvent and aqueous mixture (e.g., water) produce a partial mixture. In some embodiments, the aqueous mixture phase is separated from the organic mixture phase to provide a dosage composition. In some embodiments, the aqueous mixture phase is separated from the organic mixture phase for further processing (e.g., one or more washing steps with a second organic solvent, one or more additional separation steps, one or more evaporation steps).

In some embodiments (i.e., for PHIP-SAH procedures), the polarization step is followed by the sidearm being cleaved (e.g., via hydrolysis with an aqueous mixture) from the target molecule precursor (e.g., biorelevant imaging agent precursor) to produce a target molecule (e.g., biorelevant imaging agent) and an unbound sidearm (such as a compound of Formula IIIa, as described herein). In some embodiments, the polarization step is followed by the sidearm being cleaved by mixing the solution (which includes the first organic solvent and the polarized product, e.g., hyperpolarized biorelevant imaging agent or a pharmaceutically acceptable salt thereof) with a hydrolyzing agent, such as a base (e.g., sodium hydroxide) in an aqueous solution. In some embodiments, the first organic solvent and the aqueous mixture (e.g., water) produce a biphasic solution. In some embodiments, the first organic solvent and aqueous mixture (e.g., water) produce a biphasic solution, wherein a portion of the organic solvent is retained in the aqueous mixture. In some embodiments, the first organic solvent and aqueous mixture (e.g., water) produce a partial mixture.

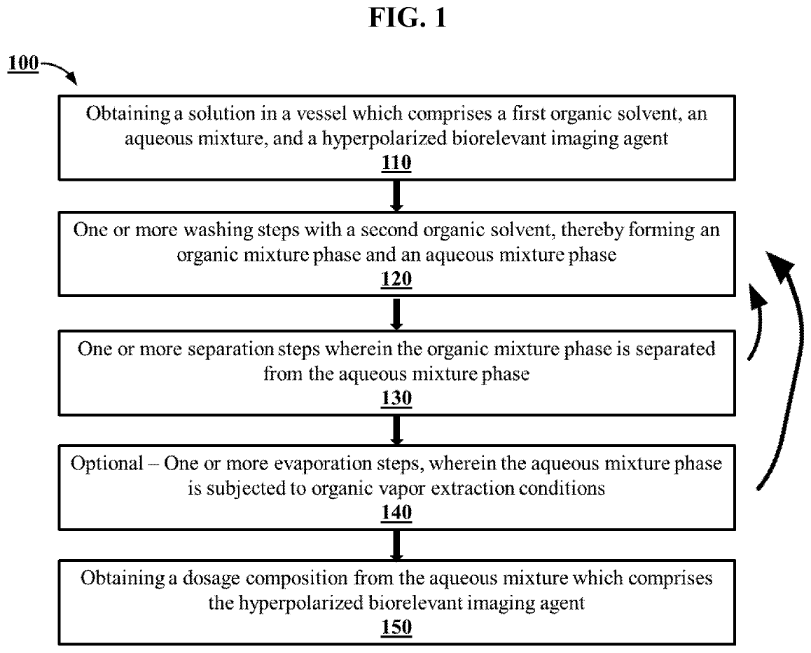

FIG. 1 presents an embodiment 100 of a multi-step liquid-liquid separation and purification process of the present disclosure. In step 110, a solution is obtained and provided in a vessel, wherein the solution comprises a first organic solvent, an aqueous mixture, a hyperpolarized biorelevant imaging agent or a pharmaceutically acceptable salt thereof, and optionally an unbonded sidearm. In some embodiments, the solution can be obtained by methods of producing a hyperpolarized biorelevant imaging agent according to the present disclosure. In step 120, the solution from step 110 is processed through one or more washing steps, wherein the solution is washed with a second organic solvent. Step 120 forms (i) an organic mixture phase comprising the first organic solvent, the second organic solvent, and the unbonded sidearm (if present), and (ii) an aqueous mixture phase comprising the aqueous mixture and the hyperpolarized biorelevant imaging agent or a pharmaceutically acceptable salt thereof. In step 130, the mixture from step 120 is processed through one or more separation steps, wherein the organic mixture phase is separated from the aqueous mixture phase, such as by either the organic mixture phase or the aqueous mixture phase being transferred into a separate vessel. Following step 130, the resulting aqueous mixture phase can be re-cycled through step 120 and/or step 130 one or more times to further process the aqueous mixture. In optional step 140, the aqueous mixture from step 120 and step 130 is optionally processed through one or more evaporation steps, wherein the aqueous mixture is subjected to organic vapor extraction conditions to evaporate at least a portion of organic solvent remaining in the aqueous mixture. In some embodiments, the organic vapor extraction conditions comprise bubbling with an inert gas, such as nitrogen gas (e.g., nitrogen gas bubbling using glass frit at 60° C. and 150 mbar absolute pressure by vacuum). Following optional step 140, the resulting aqueous mixture phase can be re-cycled through step 120, step 130, and/or step 140 one or more times to further process the aqueous mixture. In step 150, a dosage composition is obtained from the aqueous mixture from steps 120/130/140, which comprises the hyperpolarized biorelevant imaging agent or a pharmaceutically acceptable salt thereof.

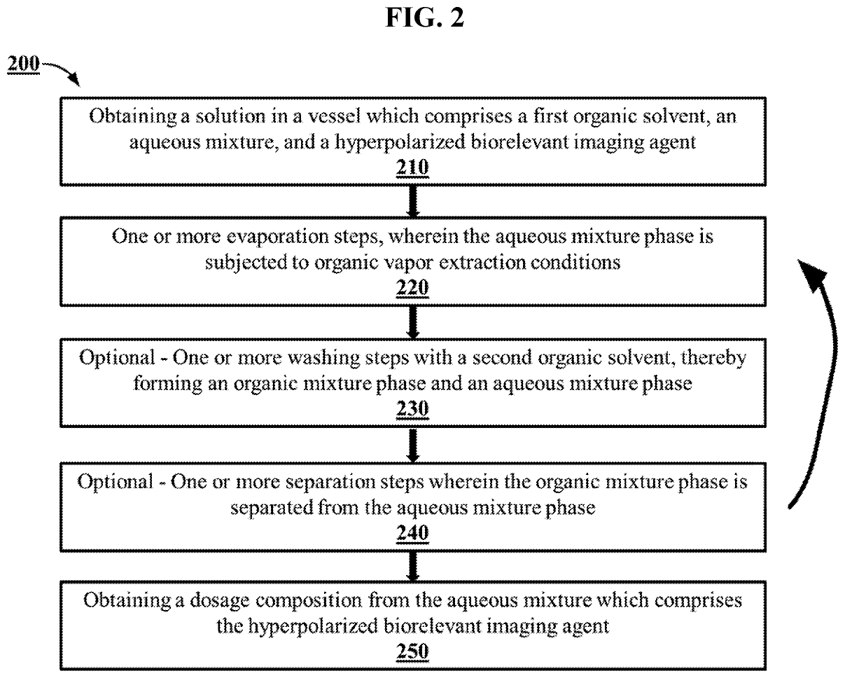

FIG. 2 presents an embodiment 200 of a multi-step liquid-liquid separation and purification process of the present disclosure. In step 210, a solution is obtained and provided in a vessel, wherein the solution comprises a first organic solvent, an aqueous mixture, a hyperpolarized biorelevant imaging agent or a pharmaceutically acceptable salt thereof, and optionally an unbonded sidearm. In some embodiments, the solution can be obtained by methods of producing a hyperpolarized biorelevant imaging agent according to the present disclosure. In step 220, the solution from step 210 is processed through one or more evaporation steps, wherein the solution is subjected to organic vapor extraction conditions to evaporate at least a portion of organic solvent remaining in the solution. In some embodiments, the organic vapor extraction conditions comprise bubbling with an inert gas, such as nitrogen gas (e.g., nitrogen gas bubbling using glass frit at 60° C. and 150 mbar absolute pressure by vacuum). In optional step 230, the resulting solution from step 220 (which comprises the aqueous mixture and the hyperpolarized biorelevant imaging agent or a pharmaceutically acceptable salt thereof) is processed through one or more washing steps, wherein the solution is washed with a second organic solvent. Step 230 forms (i) an organic mixture phase comprising at least a portion of the remaining first organic solvent, second organic solvent, and unbonded sidearm (if present), and (ii) an aqueous mixture phase comprising the aqueous mixture and the hyperpolarized biorelevant imaging agent or a pharmaceutically acceptable salt thereof. In optional step 240, the mixture from step 230 is processed through one or more separation steps, wherein the organic mixture phase is separated from the aqueous mixture phase, such as by either the organic mixture phase or the aqueous mixture phase being transferred into a separate vessel. Following step 240, the resulting aqueous mixture phase can be re-cycled through step 220, step 230, and/or step 240 one or more times to further process the aqueous mixture. In step 250, a dosage composition is obtained from the aqueous mixture from steps 220/230/240, which comprises the hyperpolarized biorelevant imaging agent or a pharmaceutically acceptable salt thereof.

In some embodiments, a target molecule precursor with a sidearm (e.g., biorelevant imaging agent precursor) is polarized (e.g., hydrogenation via the PHIP-SAH process) in a solution containing a first organic solvent. In some embodiments, the first organic solvent is chosen such that the hydrogenation of the precursor is efficient and facilitates a high spin order on the parahydrogenated protons. In some embodiments, the first solvent is chosen from acetone, ethanol, methanol, chloroform, ethyl acetate, methyl ethyl ketone, acetophenone, hexone, cyclohexanone, or cyclopentanone. In some embodiments, the first solvent is acetone. In some embodiments, the first organic solvent has a solubility in water of greater than 50 millimolar (mM) at 20° C. In some embodiments, the first organic solvent has a solubility in water of greater than 55 millimolar (mM) at 20° C. In some embodiments, the first organic solvent has a solubility in water of greater than 60 millimolar (mM) at 20° C. In some embodiments, the first organic solvent has a solubility in water of greater than 65 millimolar (mM) at 20° C. In some embodiments, the first organic solvent has a solubility in water of greater than 70 millimolar (mM) at 20° C. In some embodiments, the first organic solvent has a solubility in water of greater than 75 millimolar (mM) at 20° C. In some embodiments, the first organic solvent has a solubility in water of greater than 80 millimolar (mM) at 20° C. In some embodiments, the first organic solvent has a solubility in water of greater than 85 millimolar (mM) at 20° C. In some embodiments, the first organic solvent has a solubility in water of greater than 90 millimolar (mM) at 20° C. In some embodiments, the first organic solvent has a solubility in water of greater than 95 millimolar (mM) at 20° C. In some embodiments, the first organic solvent has a solubility in water of greater than 100 millimolar (mM) at 20° C. In some embodiments, the first organic solvent has a solubility in water of greater than 125 millimolar (mM) at 20° C. In some embodiments, the first organic solvent has a solubility in water of greater than 150 millimolar (mM) at 20° C. In some embodiments, the first organic solvent has a solubility in water of greater than 200 millimolar (mM) at 20° C. In some embodiments, the first organic solvent has a solubility in water of greater than 300 millimolar (mM) at 20° C. In some embodiments, the first organic solvent has a solubility in water of greater than 400 millimolar (mM) at 20° C. In some embodiments, the first organic solvent has a solubility in water of greater than 500 millimolar (mM) at 20° C. In some embodiments, the first organic solvent has a solubility in water at 20° C. which is greater than the solubility of chloroform in water at 20° C. In some embodiments, the first organic solvent does not comprise chloroform.

In some embodiments, a solution containing the aqueous solvent (e.g., water) and the first organic solvent undergoes one or more washing steps with a second organic solvent. In some embodiments, the second organic solvent is a biocompatible solvent. In some embodiments, the second organic solvent is a class 2 solvent according to the International Council for Harmonisation of Technical Requirements for Pharmaceuticals for Human Use (ICH) guidelines. In some embodiments, the second organic solvent comprises one or more class 2 solvents selected from: Acetonitrile, Chlorobenzene, Chloroform, Cyclohexane, Dibromomethane, 1,2-Dichloroethene, Dichloromethane, 1,2-Dimethoxyethane, N,N-Dimethylacetamide, N,N-Dimethylformamide, 1,4-Dioxane, 2-Ethoxyethanol, Ethyleneglycol, Formamide, Hexane, Methanol, 2-Methoxyethanol, Methylbutyl ketone, Methylcyclohexane, N-Methylpyrrolidone, Nitromethane, Pyridine, Sulfolane, Tetrahydrofuran, Tetralin, Toluene, 1,1,2-Trichloroethene, or Xylene. In some embodiments, the second organic solvent is a class 3 solvent according to the ICH guidelines. In some embodiments, the second organic solvent comprises one or more class 3 solvents selected from: Acetic acid, Acetone, Anisole, 1-Butanol, 2-Butanol, Butyl acetate, tert-Butylmethyl ether, Cumene, Diethyl ether, Dimethyl sulfoxide, Ethanol, Ethyl acetate, Ethyl ether, Ethyl formate, Formic acid, Heptane, Isobutyl acetate, Isopropyl acetate, Methyl acetate, 3-Methyl-1-butanol, Methylethyl ketone, Methylisobutyl ketone, 2-Methyl-1-propanol, Pentane, 1-Pentanol, 1-Propanol, 2-Propanol, or Propyl acetate. In some embodiments, the second organic solvent is a class 2 solvent or a class 3 solvent, according to ICH guidelines. In some embodiments, the second organic solvent facilitates the separation and/or extraction of one or more of the following from the aqueous solvent: the cleaved sidearm residues, the first organic solvent, and any catalyst residues (e.g., rhodium atoms, iridium atoms).

In some embodiments, the second organic solvent has a solubility in water of greater than 50 millimolar (mM) at 20° C. In some embodiments, the second organic solvent has a solubility in water of greater than 75 millimolar (mM) at 20° C. In some embodiments, the second organic solvent has a solubility in water of greater than 100 millimolar (mM) at 20° C. In some embodiments, the second organic solvent has a solubility in water of greater than 150 millimolar (mM) at 20° C. In some embodiments, the second organic solvent has a solubility in water of greater than 200 millimolar (mM) at 20° C. In some embodiments, the second organic solvent has a solubility in water of greater than 250 millimolar (mM) at 20° C. In some embodiments, the second organic solvent has a solubility in water of greater than 300 millimolar (mM) at 20° C. In some embodiments, the second organic solvent has a solubility in water of greater than 350 millimolar (mM) at 20° C. In some embodiments, the second organic solvent has a solubility in water of greater than 400 millimolar (mM) at 20° C. In some embodiments, the second organic solvent has a solubility in water of greater than 450 millimolar (mM) at 20° C. In some embodiments, the second organic solvent has a solubility in water of greater than 500 millimolar (mM) at 20° C. In some embodiments, the second organic solvent has a solubility in water of less than 500 millimolar (mM) at 20° C. In some embodiments, the second organic solvent has a solubility in water of less than 450 millimolar (mM) at 20° C. In some embodiments, the second organic solvent has a solubility in water of less than 400 millimolar (mM) at 20° C. In some embodiments, the second organic solvent has a solubility in water of less than 350 millimolar (mM) at 20° C. In some embodiments, the second organic solvent has a solubility in water of less than 300 millimolar (mM) at 20° C. In some embodiments, the second organic solvent has a solubility in water of less than 250 millimolar (mM) at 20° C. In some embodiments, the second organic solvent has a solubility in water of less than 200 millimolar (mM) at 20° C. In some embodiments, the second organic solvent has a solubility in water of less than 150 millimolar (mM) at 20° C. In some embodiments, the second organic solvent has a solubility in water of less than 100 millimolar (mM) at 20° C. In some embodiments, the second organic solvent has a solubility in water of less than 75 millimolar (mM) at 20° C. In some embodiments, the second organic solvent has a solubility in water of less than 50 millimolar (mM) at 20° C.

In some embodiments, a solution containing the aqueous solvent (e.g., water) and the first organic solvent undergoes one or more evaporation step. In some embodiments, a solution containing the aqueous solvent (e.g., water) and the first organic solvent undergoes one or more evaporation step to reduce or remove an amount of volatile organic material from the solution. In some embodiments, the evaporation step comprises flushing and/or bubbling gas (e.g., nitrogen gas) through the solution. In some embodiments, the evaporation step reduces the amount of organic solvent in the solution.

In some embodiments, the solution containing the aqueous solvent (e.g., water) and the first organic solvent undergoes: (i) one or more washing steps with a second organic solvent, (ii) one or more evaporation steps, and (iii) optionally, one or more additional processing steps (e.g., filtration, fill/finish, dilution), resulting in the production of a dosage composition (i.e., composition pharmaceutically acceptable for dosage of a subject) comprising a biorelevant imaging agent.

In some embodiments, the present disclosure presents a dosage composition (i.e., composition pharmaceutically acceptable for dosage of a subject) which comprises a biorelevant imaging agent and a pharmaceutically acceptable carrier (e.g., solvent). In some embodiments, the dosage composition comprises a level of residual organic solvents below the toxicity limits under the ICH Q3C guidance. In some embodiments, the dosage composition comprises a level of residual ICH Class 1, Class 2, or Class 3 organic solvents below the toxicity limits presented in Table 1. As used herein, “ppm” refers to the parts-per-million concentration of the residual organic solvent in the pharmaceutically acceptable carrier.

TABLE 1
ICH Toxicity Limits

| | | |
|---|---|---|
| | Permissible Daily | Limits |
| Solvent | Exposure (mg/day) | (ppm) |

Class 1: Most Toxic Solvents

| | | |
|---|---|---|
| Benzene | — | 2 |
| Carbon tetrachloride | — | 4 |
| 1,2-Dichloroethane | — | 5 |
| 1,1-Dichloroethene | — | 8 |
| 1,1,1-Trichloroethane | — | 1500 |

Class 2: Less Toxic Solvents

| | | |
|---|---|---|
| Acetonitrile | 4.1 | 410 |
| Chlorobenzene | 3.6 | 360 |
| Chloroform | 0.6 | 60 |
| Cyclohexane | 38.8 | 3880 |
| 1,2-Dichloroethene | 18.7 | 1870 |
| Dichloromethane | 6.0 | 600 |
| 1,2-Dimethoxyethane | 1.0 | 100 |
| N,N-Dimethylacetamide | 10.9 | 1090 |
| N,N-Dimethylformamide | 8.8 | 880 |
| 1,4-Dioxane | 3.8 | 380 |
| 2-Ethoxyethanol | 1.6 | 160 |
| Ethylene glycol | 6.2 | 620 |
| Formamide | 2.2 | 220 |
| Hexane | 2.9 | 290 |
| Methanol | 30.0 | 3000 |
| 2-Methoxyethanol | 0.5 | 50 |
| Methylbutyl ketone | 0.5 | 50 |
| Methylcyclohexane | 11.8 | 1180 |
| N-Methylpyrrolidone | 15.3 | 530 |
| Nitromethane | 0.5 | 50 |
| Pyridine | 2.0 | 200 |
| Sulfolane | 1.6 | 160 |
| Tetrahydrofuran | 27.2 | 720 |
| Tetralin | 1.0 | 100 |
| Toluene | 8.9 | 890 |
| 1,1,2-Trichloroethene | 0.8 | 80 |
| Xylene | 21.7 | 2170 |

Class 3: Safest Solvents

| | | |
|---|---|---|
| Acetic acid | 50 | 5000 |
| Acetone | 50 | 5000 |
| Anisole | 50 | 5000 |
| 1-Butanol | 50 | 5000 |
| 2-Butanol | 50 | 5000 |
| Butyl acetate | 50 | 5000 |
| tert-Butylmethyl ether | 50 | 5000 |
| Cumene | 50 | 5000 |
| Diethyl ether | 50 | 5000 |
| Dimethyl sulfoxide | 50 | 5000 |
| Ethanol | 50 | 5000 |
| Ethyl acetate | 50 | 5000 |
| Ethyl ether | 50 | 5000 |
| Ethyl formate | 50 | 5000 |
| Formic acid | 50 | 5000 |
| Heptane | 50 | 5000 |
| Isobutyl acetate | 50 | 5000 |
| Isopropyl acetate | 50 | 5000 |
| Methyl acetate | 50 | 5000 |
| 3-Methyl-1-butanol | 50 | 5000 |
| Methylethyl ketone | 50 | 5000 |
| Methylisobutyl ketone | 50 | 5000 |
| 2-Methyl-1-propanol | 50 | 5000 |
| Pentane | 50 | 5000 |
| 1-Pentanol | 50 | 5000 |
| 1-Propanol | 50 | 5000 |
| 2-Propanol | 50 | 5000 |
| Propyl acetate | 50 | 5000 |

### Systems

In some embodiments, the present disclosure describes a system for implementing the methods of the present disclosure. In some embodiments, the present disclosure describes a system for implementing the methods of the present disclosure, wherein the system comprises a first vessel, and a second vessel fluidly connected with the first vessel. In some embodiments, the present disclosure describes a system for implementing the methods of the present disclosure, wherein the system comprises a first vessel, and a second vessel fluidly connected with the first vessel through a first fluid transfer element. In some embodiments, the present disclosure describes a system for implementing the methods of the present disclosure, wherein the system comprises a first vessel, a second vessel fluidly connected with the first vessel, and a third vessel fluidly connected with the second vessel. In some embodiments, the present disclosure describes a system for implementing the methods of the present disclosure, wherein the system comprises a first vessel, a second vessel fluidly connected with the first vessel through a first fluid transfer element, and a third vessel fluidly connected with the second vessel through a second fluid transfer element.

In some embodiments, the system further comprises a magnetic guide system. In some embodiments, the system further comprises a magnetic guide system which provides a defined magnetic field over at least a portion of the system. In some embodiments, the system further comprises a magnetic guide system which comprises one or more solenoids which provide the defined magnetic field. In some embodiments, the system further comprises a magnetic guide system which provides a defined magnetic field over the first vessel, second vessel, and/or optional third vessel. In some embodiments, the system further comprises a magnetic guide system which provides a defined magnetic field over the first fluid transfer element and/or optional second fluid transfer element. In some embodiments, the system further comprises a magnetic guide system which provides a defined magnetic field over the entire system. In some embodiments, the defined magnetic field prevents undesired hyperpolarization loss in the solution during implementation of the methods of the present disclosure.

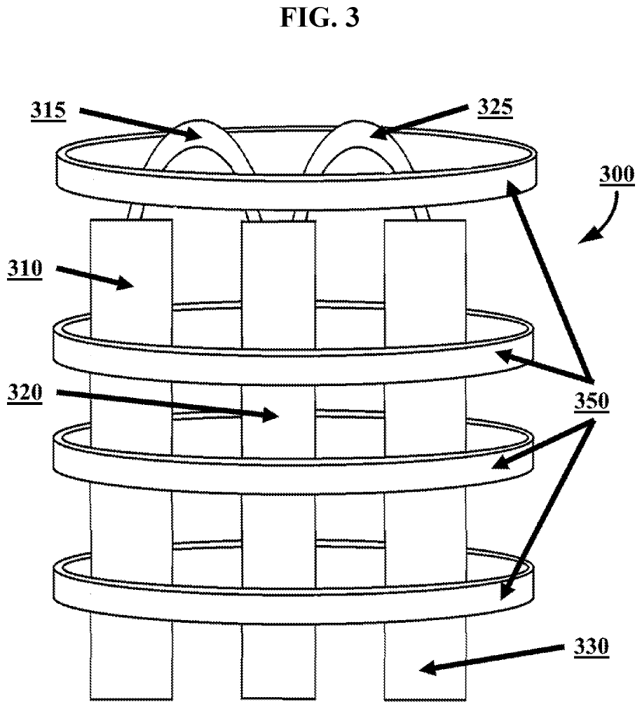

FIG. 3 presents an embodiment 300 of a multi-step liquid-liquid separation and purification system of the present disclosure. System 300 comprises a first vessel 310, a second vessel 320, and a third vessel 330. First vessel 310 is fluidly connected with second vessel 320 through a first fluid transfer element 315. Second vessel 320 is fluidly connected with third vessel 330 through a second fluid transfer element 325.

System 300 optionally comprises magnetic guide system 350, which provides a defined magnetic field over at least a portion of the system 300. In some embodiments, the magnetic guide system 350 comprises one or more solenoids which provide the defined magnetic field. In some embodiments, the magnetic guide system 350 provides a defined magnetic field over first vessel 310, second vessel 320, and/or third vessel 330. In some embodiments, the magnetic guide system 350 provides a defined magnetic field over the first fluid transfer element 315 and/or second fluid transfer element 325. In some embodiments, the magnetic guide system 350 provides a defined magnetic field over the entire system 300. In some embodiments, the defined magnetic field prevents undesired hyperpolarization loss in the solution during implementation of the methods of the present disclosure.

In some embodiments, system 300 can be used in implementing a multi-step liquid-liquid separation and purification process of the present disclosure. In some embodiments, a solution is provided in vessel 310, wherein the solution comprises a first organic solvent, an aqueous mixture, a hyperpolarized biorelevant imaging agent or a pharmaceutically acceptable salt thereof, and optionally an unbonded sidearm. In some embodiments, the solution can be obtained by methods of producing a hyperpolarized biorelevant imaging agent according to the present disclosure, including embodiments where the methods of producing a hyperpolarized biorelevant imaging agent are completed, in part or as a whole, within vessel 310. The solution in vessel 310 is processed through one or more washing steps, wherein the solution is washed with a second organic solvent, which forms with vessel 310: (i) an organic mixture phase comprising the first organic solvent, the second organic solvent, and the unbonded sidearm (if present), and (ii) an aqueous mixture phase comprising the aqueous mixture and the hyperpolarized biorelevant imaging agent or a pharmaceutically acceptable salt thereof. The resulting mixture in vessel 310 is processed through one or more separation steps, wherein the aqueous mixture phase is separated from the organic mixture phase by transferring the aqueous mixture phase into vessel 320 using the first fluid transfer element 315. The resulting aqueous solution in vessel 320 is processed through one or more additional washing steps, wherein the solution is again washed with a second organic solvent, which forms with vessel 320: (i) an organic mixture phase comprising at least a portion of the remaining first organic solvent and the second organic solvent, and (ii) an aqueous mixture phase comprising the aqueous mixture and the hyperpolarized biorelevant imaging agent or a pharmaceutically acceptable salt thereof. The resulting mixture in vessel 320 is processed through one or more additional separation steps, wherein the aqueous mixture phase is separated from the organic mixture phase by transferring the aqueous mixture phase into vessel 330 using the second fluid transfer element 325. The resulting aqueous mixture in vessel 330 is then processed through one or more evaporation steps, wherein the aqueous mixture is subjected to organic vapor extraction conditions to evaporate at least a portion of organic solvent remaining in the aqueous mixture. In some embodiments, the organic vapor extraction conditions comprise bubbling with an inert gas, such as nitrogen gas (e.g., nitrogen gas bubbling using glass frit at 60° C. and 150 mbar absolute pressure by vacuum). In some embodiments, a dosage composition is obtained from the aqueous mixture in vessel 330 after one or more evaporation steps, wherein the dosage composition comprises the hyperpolarized biorelevant imaging agent or a pharmaceutically acceptable salt thereof.

In some embodiments, first vessel 310, second vessel 320, and/or third vessel 330 can each, independently, comprise a commercially available or self-designed vessel which is made of chemical and physical (temperature, pressure) suitable material, such as plastics (e.g., polyetheretherketone [PEEK], polytetrefluoroethylene [PTFE], fluorinated ethylene propylene [FEP], ethylene tetrafluoroethylene [ETFE], perfluoroelastomer [FFKM], polypropylene [PP], polyethylene [PE]), glass, and non-magnetic metals/alloys. In some embodiments, first vessel 310, second vessel 320, and/or third vessel 330 can each, independently, comprise a centrifuge tube, optionally between 10 ml and 1000 ml, and optionally made of polypropylene (PP). In some embodiments, the first vessel 310 and/or second vessel 320 can comprise two chambers of a single part produced in an injection molded process using the plastics (PP, PEEK or other compatible molded plastic).

In some embodiments, first fluid transfer element 315 and/or second fluid transfer element 325 can, independently, comprise: (a) at least one valve component, (b) at least one tubing component, (c) at least one tubing connector component, or (d) any combination of (a), (b), and (c). In some embodiments, the valve component is: manual actuated (e.g., stopcock valve), electrically actuated (e.g., solenoid, motor), pressure actuated (e.g., pneumatic), or a combination thereof. In some embodiments, the valve components comprises a 3/2-way Bürkert Type 6724 solenoid. In some embodiments, the tubing component comprises a microfluidic tubing (e.g., standard tubing for laboratory use). In some embodiments, the tubing dimensions are 0.5 mm to 4 mm outer diameter (e.g., 1.5 mm) and 0.25-1 mm inner diameter (e.g., 0.5 mm). In some embodiments, the tubing component comprises a commercially available or self-designed tubing which is made of chemical and physical (temperature, pressure) suitable material, such as plastics (e.g., polyetheretherketone [PEEK], polytetrefluoroethylene [PTFE], fluorinated ethylene propylene [FEP], ethylene tetrafluoroethylene [ETFE], perfluoroelastomer [FFKM], polypropylene [PP], polyethylene [PE]), glass, and non-magnetic metals/alloys. In some embodiments, the tubing connector component comprises microfluidic fittings (e.g., standard fittings for laboratory use). In some embodiments, the tubing connector component comprises ¼″-28 fittings with a flat bottom ferrule as sealing. In some embodiments, the tubing connector component comprises a molded plastic tube connection or orifice between two chambers of the same part.

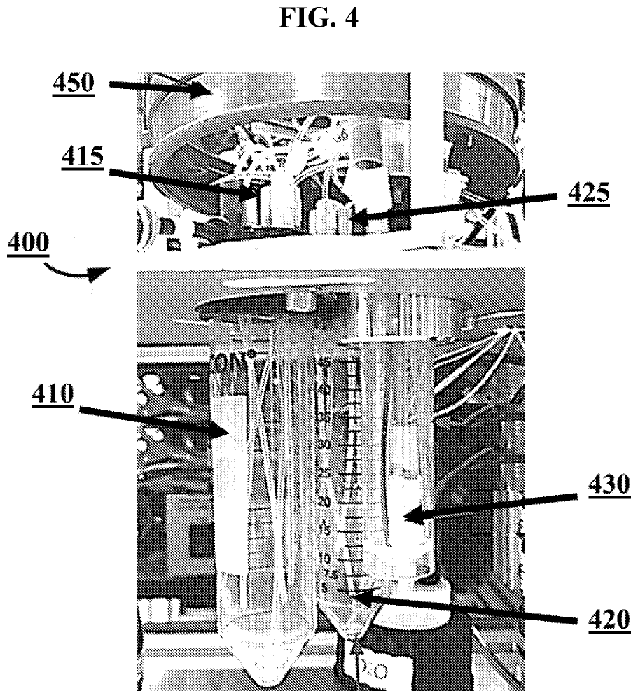

FIG. 4 presents an embodiment 400 of a multi-step liquid-liquid separation and purification system of the present disclosure. System 400 comprises a first vessel 410, a second vessel 420, and a third vessel 430. First vessel 410 is fluidly connected with second vessel 420 through a first fluid transfer element 415. Second vessel 420 is fluidly connected with third vessel 430 through a second fluid transfer element 425.

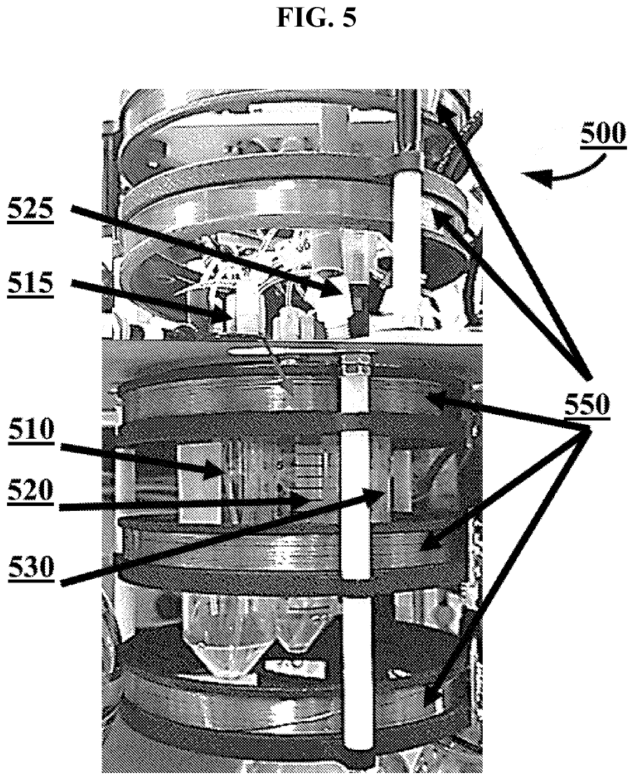

FIG. 5 presents an embodiment 500 of a multi-step liquid-liquid separation and purification system of the present disclosure. System 500 comprises a first vessel 510, a second vessel 520, and a third vessel 530. First vessel 510 is fluidly connected with second vessel 520 through a first fluid transfer element 515. Second vessel 520 is fluidly connected with third vessel 530 through a second fluid transfer element 525. System 500 also optionally comprises magnetic guide system 550, which provides a defined magnetic field over at least a portion of the system 500. In some embodiments, the magnetic guide system 550 can comprise one or more of: permanent magnets, electromagnets, ferromagnetic-core (e.g, iron-core) electromagnets, ferrimagnetic-core (e.g., ferrite-core) electromagnets, superconducting magnets, solenoids, Helmholtz coils, Maxwell coils, birdcage coils, crossed coils, saddle coils, paired saddle coils, dipolar magnets, or any combination thereof. In some embodiments, the magnetic guide system 550 is configured to generate a magnetic field or a magnetic field gradient in the vicinity of all or a portion of the system 500. For instance, in some embodiments, the magnetic guide system 550 is configured to generate the magnetic field or the magnetic field gradient in the vicinity of any one, two, three, four, five, or six of the first vessel 510, the first fluid transfer element 515, the second vessel 520, the second fluid transfer element 525, and the third vessel 530. In some embodiments, the magnetic field guide 550 is configured to mitigate loss of polarization due to spurious magnetic fields associated with any one, two, three, four, five, or six of the first vessel 510, the first fluid transfer element 515, the second vessel 520, the second fluid transfer element 525, and the third vessel 530 or spurious magnetic fields associated with the environment surrounding the system 500.

In some embodiments, the magnetic field has an average magnetic field strength of at least about 10 nT, 20 nT, 30 nT, 40 nT, 50 nT, 60 nT, 70 nT, 80 nT, 90 nT, 100 nT, 200 nT, 300 nT, 400 nT, 500 nT, 600 nT, 700 nT, 800 nT, 90 nT, 1 μT, 2 μT, 3 μT, 4 μT, 5 μT, 6 μT, 7 μT, 8 μT, 9 μT, 10 μT, 20 μT, 30 μT, 40 μT, 50 μT, 60 μT, 70 μT, 80 μT, 90 μT, 100 μT, 200 μT, 300 μT, 400 μT, 500 μT, 600 μT, 700 μT, 800 μT, 900 μT, 1 mT, 2 mT, 3 mT, 4 mT, 5 mT, 6 mT, 7 mT, 8 mT, 9 mT, 10 mT, 20 mT, 30 mT, 40 mT, 50 mT, 60 mT, 70 mT, 80 mT, 90 mT, 100 mT, 200 mT, 300 mT, 400 mT, 500 mT, 600 mT, 700 mT, 800 mT, 900 mT, 1,000 mT, or more, at most about 1,000 mT, 900 mT, 800 mT, 700 mT, 600 mT, 500 mT, 400 mT, 300 mT, 200 mT, 100 mT, 90 mT, 80 mT, 70 mT, 60 mT, 50 mT, 40 mT, 30 mT, 20 mT, 10 mT, 9 mT, 8 mT, 7 mT, 6 mT, 5 mT, 4 mT, 3 mT, 2 mT, 1 mT, 900 μT, 800 μT, 700 μT, 600 μT, 500 μT, 400 μT, 300 μT, 200 μT, 100 μT, 90 μT, 80 μT, 70 μT, 60 μT, 50 μT, 40 μT, 30 μT, 20 μT, 10 μT, 9 μT, 8 μT, 7 μT, 6 μT, 5 μT, 4 μT, 3 μT, 2 μT, 1 μT, 900 nT, 800 nT, 700 nT, 600 nT, 500 nT, 400 nT, 300 nT, 200 nT, 100 nT, 90 nT, 80 nT, 70 nT, 60 nT, 50 nT, 40 nT, 30 nT, 20 nT, 100 nT, or an average magnetic field strength that is within a range defined by any two of the preceding values.

### Clinically Relevant Purities

Consistent with disclosed embodiments, steps the methods and systems described herein can separate the hyperpolarized biorelevant imaging agent from other substances in the original solution (e.g., catalysts, the original solvent(s), reaction products, or the like). For example, most of the hydrogenation catalyst present in the original solution can removed from the dosage composition. In some embodiments, the dosage composition can retain at most about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, or less of the hydrogenation catalyst, at least about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, or more of the hydrogenation catalyst, or an amount of the hydrogenation catalyst that is within a range defined by any two of the preceding values. Similarly, the dosage composition can retain at most about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, or less of the cleavage byproducts (e.g., the sidearm or other residues of the cleavage), at least about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, or more of the cleavage byproducts, or an amount of the cleavage byproducts that is within a range defined by any two of the preceding values.

In some embodiments, the methods and systems described herein produce dosage compositions in which the concentration of the hyperpolarized biorelevant imaging is at least about 100 mM, 150 mM, 200 mM, 250 mM, 300 mM, 350 mM, 400 mM, 450 mM, 500 mM, or more, at most about 500 mM, 450 mM, 400 mM, 350 mM, 300 mM, 250 mM, 200 mM, 150 mM, 100 mM, or less, or within a range defined by any two of the preceding values.

In some embodiments, the methods and systems described herein produce dosage compositions in which the polarization of the hyperpolarized biorelevant imaging is at least about 1%, 2%, 3%, 4%, 5%, 6%, 7%, 8%, 9%, 10%, 15%, 20%, 25%, 30%, 40%, 45%, 50%, more, at most about 50%, 45%, 40%, 35%, 30%, 25%, 20%, 15%, 10%, 9%, 8%, 7%, 6%, 5%, 4%, 3%, 2%, 1%, or less, or polarization that is within a range defined by any two of the preceding values. For example, in some embodiments, the methods and systems described herein produce dosage compositions in which the polarization of the hyperpolarized biorelevant imaging is between 10% and 15%, between 10% and 20%, between 10% and 25%, between 10% and 30%, between 10% and 35%, between 10% and 40%, between 10% and 45%, between 10% and 50%, between 15% and 20%, between 15% and 25%, between 15% and 30%, between 15% and 35%, between 15% and 40%, between 15% and 45%, between 15% and 50%, between 20% and 25%, between 20% and 30%, between 20% and 35%, between 20% and 40%, between 20% and 45%, between 20% and 50%, between 25% and 30%, between 25% and 35%, between 25% and 40%, between 25% and 45%, between 25% and 50%, between 30% and 35%, between 30% and 40%, between 30% and 45%, between 30% and 50%, between 35% and 40%, between 35% and 45%, between 35% and 50%, between 40% and 45%, between 40% and 50%, or between 45% and 50%.

In some embodiments, the methods and systems described herein produce dosage compositions in which the concentration of catalysts, the precursor, or the cleavage byproducts may each be at most about 1 μM, 900 nanomolar (nM), 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less, at least about 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, or more, or within a range defined by any two of the preceding values. the methods and systems described herein produce dosage compositions in which the purity of the hyperpolarized biorelevant imaging is at least about 90%91%, 92%, 93%, 94%, 95%, 96%, 97%, 98%, 99%, or more, at most about 99%, 98%, 97%, 96%, 95%, 94%, 93%, 92%, 91%, 90%, or less, or within a range defined by any two of the preceding values. In some embodiments, at least a fraction of the hyperpolarized compounds is separated from the cleaved sidearms, or other reaction byproducts, if such exist.

### Transportation

Consistent with disclosed embodiments, polarization transfer and use of the biorelevant imaging agent can occur at different locations. In some embodiments, the dosage composition is transported to another location. In some embodiments, the dosage composition is transported to another location. The disclosed embodiments are not necessarily limited to any particular transport distance or duration. Instead, a maximum distance or duration can be determined based on the target molecule, the original degree or polarization, the required final degree of polarization, and the transport conditions. In some embodiments, the dosage composition is transported at least one meter in a suitable transportation device.

Consistent with disclosed embodiments, a transportation device can be configured to transport samples of the precursor or biorelevant imaging agent. The transportation device can be arranged and configured for transporting one or more samples (e.g., one or more dosage compositions) simultaneously. The transportation device can include a transport chamber configured to receive the one or more samples. The transportation device can be configured to maintain the transport chamber within a predetermined temperature range and a predetermined magnetic field strength. The transportation device can be configured to maintain the one or more samples in a magnetic field of at least about 10 G, 20 G, 30 G, 40 G, 50 G, 60 G, 70 G, 80 G, 90 G, 100 G, 200 G, 300 G, 400 G, 500 G, 600 G, 700 G, 800 G, 900 G, 1,000 G, or more, at most about 1,000 G, 900 G, 800 G, 700 G, 600 G, 500 G, 400 G, 300 G, 200 G, 100 G, 90 G, 80 G, 70 G, 60 G, 50 G, 40 G, 30 G, 20 G, 10 G, or less, or within a magnetic field that is within a range defined by any two of the previous values.

A permanent magnet or an electromagnet included in the transportation device can provide the magnetic field. In some embodiments, the permanent magnet or electromagnet is shielded to reduce the strength of the magnetic field outside the transportation device. The transportation device can also include a cooling system. The cooling system can be configured to maintain samples at a predetermined temperate or within a predetermined range of temperatures during transport. For example, the cooling system can be configured to maintain the samples at a temperature below 270 K, below 80 K, or below 4 K. In some embodiments, the transportation device is configured to maintain the samples at approximately the temperature of liquid nitrogen. The transportation device can include insulation between the cooling system and the exterior of the transportation device, to minimize heat exchange with the external environment. In some embodiments, the cooling system is configured to maintain the temperature of the samples using a cold gas flow. In some embodiments, the cooling system is configured to maintain the temperature of the samples using a liquid coolant. In some embodiments, the transportation device includes a Dewar to provide cooling of the samples. In order to distribute the hyperpolarized samples also across large distances, the container can be transported by standard transportation vehicles, such as planes, trains, trucks, cars and ships.

In some embodiments, the dosage composition containing the hyperpolarized biorelevant imaging agent is transported in the transportation device. In some embodiments the relaxation time of the hyperpolarized biorelevant imaging agent in the transportation device at least about 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, or more, at most about 10 hours, 9 hours, 8 hours, 7 hours, 6 hours, 5 hours, 4 hours, 3 hours, 2 hours, 1 hour, 50 minutes, 40 minutes, 30 minutes, 20 minutes, 10 minutes, 9 minutes, 8 minutes, 7 minutes, 6 minutes, 5 minutes, 4 minutes, 3 minutes, 2 minutes, 1 minute, or less, or a relaxation time that is within a range defined by any two of the preceding values.

### Generation of Polarized Biorelevant Imaging Agents

Described herein is a first exemplary process for generating polarized biorelevant imaging agents, in accordance with various embodiments. In some embodiments, the first process comprises providing a composition comprising a compound of Formula Ia. In some embodiments, the compound of Formula Ia comprises: a Z group comprising: (i) a carbon-carbon double bond (—C═C—) which is substituted to include ¹H (proton), 2H (deuterium), or a combination thereof or (ii) a carbon-carbon triple bond (—C≡C—), as described herein; an R₁ group comprising a PHIP transfer moiety descried herein; an R₂ group comprising an optionally substituted hydrocarbon, alkoxy group, primary amine, secondary amine, or tertiary amine, or a solubilizing moiety, as described herein; and an R₃ group comprising a biorelevant imaging agent, as described herein.

In some embodiments, the double bond or the triple bond in the compound of Formula Ia is hydrogenated with parahydrogen to form a parahydrogenated derivative of the compound of Formula Ia, wherein the parahydrogenated derivative is a compound having the structure of Formula IIa. In some embodiments, the compound of Formula IIa comprises: a Z′ which is: (i) a parahydrogenated carbon-carbon single bond (—CH*—CH*—) which is substituted to include ¹H (proton), ²H (deuterium), or a combination thereof, or (ii) a parahydrogenated carbon-carbon double bond (—CH*═CH*—) which is substituted to include ¹H (proton), 2H (deuterium), or a combination thereof, wherein H* is a hydrogen having a spin order derived from parahydrogen; an R₁ group comprising a PHIP transfer moiety, as described herein; an R₂ group comprising an optionally substituted hydrocarbon, alkoxy group, primary amine, secondary amine, or tertiary amine, or a solubilizing moiety, as described herein; and an R₃ group comprising a biorelevant imaging agent, as described herein. In some embodiments, the compound of Formula Ia is hydrogenated with parahydrogen using a hydrogenation process described herein.

In some embodiments, a polarization transferring waveform is applied to transfer nuclear spin order from at least one H* in the sidearm of the compound of Formula IIa to any non-hydrogen nuclear spin in the biorelevant imaging agent of the compound of Formula IIa, as described herein, thereby forming a derivative of the compound of Formula IIa having a hyperpolarized biorelevant imaging agent. In some embodiments, the nuclear spin order is transferred using any polarization transfer process described herein.

Described herein is a second exemplary process for generating polarized biorelevant imaging agents, in accordance with various embodiments of the present disclosure. In some embodiments, the second process comprises providing a composition comprising a compound of Formula IIa. In some embodiments, Formula IIa comprises a Z′ group which is: (i) a parahydrogenated carbon-carbon single bond (—CH*—CH*—) which is substituted to include ¹H (proton), ²H (deuterium), or a combination thereof, or (ii) a parahydrogenated carbon-carbon double bond (—CH*═CH*—) which is substituted to include ¹H (proton), ²H (deuterium), or a combination thereof, as described herein, wherein H* is a hydrogen having a spin order derived from parahydrogen; an R₁ group comprising a PHIP transfer moiety, as described herein; an R₂ group comprising an optionally substituted hydrocarbon, alkoxy group, primary amine, secondary amine, or tertiary amine, or a solubilizing moiety, as described herein; and an R₃ group comprising a biorelevant imaging agent, as described herein.

In some embodiments, a polarization transferring waveform is applied to the transfer nuclear spin order from at least one H* in the sidearm of the compound of Formula IIa to any non-hydrogen nuclear spin in the biorelevant imaging agent of the compound of Formula IIa, as descried herein, thereby forming a derivative of the compound of Formula IIa having a hyperpolarized biorelevant imaging agent.

In some embodiments, the derivative compound of Formula IIa is hydrolyzed to form a composition comprising a hyperpolarized biorelevant imaging agent and a separate sidearm compound of Formula IIIa. In some embodiments, the compound of Formula IIIa comprises a Z″ which is: (i) a parahydrogenated carbon-carbon single bond (—CH*—CH*—) which is substituted to include ¹H (proton), 2H (deuterium), or a combination thereof, or (ii) a parahydrogenated carbon-carbon double bond (—CH*═CH*—) which is substituted to include ¹H (proton), 2H (deuterium), or a combination thereof, as described herein; an R₁′ group comprising a parahydrogen induced polarization (PHIP) transfer moiety, as described herein; and an R₂ group comprising an optionally substituted hydrocarbon, alkoxy group, primary amine, secondary amine, or tertiary amine, or a solubilizing moiety, as described herein.

In some embodiments, the hyperpolarized biorelevant imaging agent is washed one or more times with an organic solvent. In some embodiments, the non-hydrogen nuclear spin in the biorelevant imaging agent has a non-hydrogen spin polarization after the washing step of at least about 1%, 2%, 3%, 4%, 5%, 6%, 7%, 8%, 9%, 10%, 15%, 20%, 25%, 30%, 35%, 40%, 45%, 50%, or more, at most about 50%, 45%, 40%, 35%, 30%, 25%, 20%, 15%, 10%, 9%, 8%, 7%, 6%, 5%, 4%, 3%, 2%, 1%, or less, or a non-hydrogen spin polarization that is within a range defined by any two of the preceding values.

In some embodiments, the first or second process comprises one or more additional steps or operations. In some embodiments, the first or second process omits one or more steps or operations. In some embodiments, one or more steps or operations of the first or second process are combined. In some embodiments, all steps or operations of the first or second process are combined to yield a complete process for generating a hyperpolarized imaging agent from a precursor having the structure of Formula Ia.

Described herein is a third exemplary process for generating polarized biorelevant imaging agents, in accordance with various embodiments. In the example shown, the process comprises providing a composition comprising a compound of Formula Ib. In some embodiments, the compound of Formula In comprises a Z group comprising an ethynyl (—C≡C—) group, an optionally substituted prop-2-ynyl (—C—C≡C—) group, an optionally substituted ethenyl (—C≡C—) group, an optionally substituted prop-2-enyl (—C—C═C—) group, or an optionally substituted but-3-enyl (—C—C—C═C—) group, as described herein, an R₂ group comprising an optionally substituted hydrocarbon group, alkyl group, cyclic alkyl group, aryl group, carboxyl group, keto group, or a solubilizing moiety, as described herein, and an R₃ group comprising an acyl derivative of a biorelevant imaging agent, as described herein.

In some embodiments, the double bond or the triple bond in the compound of Formula Ib is hydrogenated with parahydrogen to form a parahydrogenated derivative of the compound of Formula Ib, wherein the parahydrogenated derivative is a compound having the structure of Formula IIb. In some embodiments, the compound of Formula IIb comprises a Z′ group comprising a parahydrogenated ethenyl (—CH*═CH*—) group, an optionally substituted parahydrogenated prop-2-enyl (—C—CH*═CH*—) group, an optionally substituted parahydrogenated ethanyl (—CH*—CH*—) group, an optionally substituted parahydrogenated propanyl (—C—CH*—CH*—) group, or an optionally substituted parahydrogenated butanyl (—C—C—CH*═CH*—) group, wherein H* is a hydrogen having a spin order derived from parahydrogen, as described herein, an R₂ group comprising an optionally substituted hydrocarbon group, alkyl group, cyclic alkyl group, aryl group, carboxyl group, keto group, or alkoxy group or a solubilizing moiety, as described herein, and an R₃ group comprising an acyl derivative of a biorelevant imaging agent, as described herein. In some embodiments, the compound of Formula Ib is hydrogenated with parahydrogen using a hydrogenation process described herein.

In some embodiments, a polarization transferring waveform is applied to transfer nuclear spin order from at least one H* in the sidearm of the compound of Formula IIb to any non-hydrogen nuclear spin in the acyl derivative of the biorelevant imaging agent of the compound of Formula IIb, as described herein, thereby forming a derivative of the compound of Formula IIb having a hyperpolarized acyl derivative of the biorelevant imaging agent. In some embodiments, the nuclear spin order is transferred using any polarization transfer process described herein.

Described herein is a fourth exemplary process for generating polarized biorelevant imaging agents, in accordance with various embodiments of the present disclosure. In the example shown, the process comprises providing a composition comprising a compound of Formula IIb. In some embodiments, Formula IIb comprises a Z′ group comprising a parahydrogenated ethenyl (—CH*═CH*—) group, an optionally substituted parahydrogenated prop-2-enyl (—C—CH*═CH*—) group, an optionally substituted parahydrogenated ethanyl (—CH*—CH*—) group, an optionally substituted parahydrogenated propanyl (—C—CH*—CH*—) group, or an optionally substituted parahydrogenated butanyl (—C—C—CH*═CH*—) group, as described herein, wherein H* is a hydrogen having a spin order derived from parahydrogen, an R₂ group comprising an optionally substituted hydrocarbon group, alkyl group, cyclic alkyl group, aryl group, carboxyl group, keto group, or alkoxy group or a solubilizing moiety, as described herein, and an R₃ group comprising an acyl derivative of a biorelevant imaging agent, as described herein.

In some embodiments, a polarization transferring waveform is applied to the transfer nuclear spin order from at least one H* in the sidearm of the compound of Formula IIb to any non-hydrogen nuclear spin in the acyl derivative of the biorelevant imaging agent of the compound of Formula IIb, as descried herein, thereby forming a derivative of the compound of Formula IIb having a hyperpolarized acyl derivative of the biorelevant imaging agent.

In some embodiments, the derivative compound of Formula IIb is hydrolyzed to form a composition comprising a hyperpolarized biorelevant imaging agent and a separate sidearm compound of Formula IIIb. In some embodiments, the compound of Formula IIIb comprises a Z″ group comprising a parahydrogenated ethenyl (—CH*═CH*—) group, an optionally substituted parahydrogenated prop-2-enyl (—C—CH*═CH*—) group, an optionally substituted parahydrogenated ethanyl (—CH*—CH*—) group, an optionally substituted parahydrogenated propanyl (—C—CH*—CH*—) group, or an optionally substituted parahydrogenated butanyl (—C—C—CH*═CH*—) group, as described herein, and an R₂ group comprising an optionally substituted hydrocarbon group, alkyl group, cyclic alkyl group, aryl group, carboxyl group, keto group, or alkoxy group or a solubilizing moiety, as described herein.

In some embodiments, the hyperpolarized biorelevant imaging agent is washed one or more times with an organic solvent. In some embodiments, the non-hydrogen nuclear spin in the biorelevant imaging agent has a non-hydrogen spin polarization after the washing step of at least about 1%, 2%, 3%, 4%, 5%, 6%, 7%, 8%, 9%, 10%, 15%, 20%, 25%, 30%, 35%, 40%, 45%, 50%, or more, at most about 50%, 45%, 40%, 35%, 30%, 25%, 20%, 15%, 10%, 9%, 8%, 7%, 6%, 5%, 4%, 3%, 2%, 1%, or less, or a non-hydrogen spin polarization that is within a range defined by any two of the preceding values.

In some embodiments, the third or fourth process comprises one or more additional steps or operations. In some embodiments, the third or fourth process omits one or more steps or operations. In some embodiments, one or more steps or operations of the third or fourth process are combined. In some embodiments, all steps or operations of the third or fourth process are combined to yield a complete process for generating a hyperpolarized imaging agent from a precursor having the structure of Formula Ib.

### Enumerated Embodiments

Non-limiting embodiments of the foregoing disclosed herein include:

Enumerated Embodiment 1.A method for producing a dosage composition comprising a hyperpolarized biorelevant imaging agent or a pharmaceutically acceptable salt thereof, the method comprising: —obtaining a solution in a vessel, wherein the solution comprises a first organic solvent, an aqueous mixture, the hyperpolarized biorelevant imaging agent or a pharmaceutically acceptable salt thereof, and optionally an unbonded sidearm; —one or more washing steps, wherein the solution is washed with a second organic solvent, thereby forming: (i) an organic mixture phase comprising the first organic solvent, the second organic solvent, and the optional unbonded sidearm, and (ii) an aqueous mixture phase comprising the aqueous mixture and the hyperpolarized biorelevant imaging agent or a pharmaceutically acceptable salt thereof; —one or more separation steps wherein the organic mixture phase is separated from the aqueous mixture phase, and either the organic mixture phase or the aqueous mixture phase is transferred into a separate vessel; and—obtaining a dosage composition from the aqueous mixture which comprises the hyperpolarized biorelevant imaging agent or a pharmaceutically acceptable salt thereof.

Enumerated Embodiment 2. The method of Embodiment 1, wherein the method further comprises one or more evaporation steps, wherein the aqueous mixture phase is subjected to organic vapor extraction conditions to evaporate at least a portion of organic solvent remaining in the aqueous mixture.

Enumerated Embodiment 3.A method for producing a dosage composition comprising a hyperpolarized biorelevant imaging agent or a pharmaceutically acceptable salt thereof, the method comprising: —obtaining a solution in a vessel, wherein the solution comprises a first organic solvent, an aqueous mixture, the hyperpolarized biorelevant imaging agent or a pharmaceutically acceptable salt thereof, and optionally an unbonded sidearm; —one or more evaporation steps, wherein the solution is subjected to organic vapor extraction conditions to evaporate a portion of organic solvent, and optional unbonded sidearm, from the solution, thereby providing an aqueous mixture phase comprising the aqueous mixture and the hyperpolarized biorelevant imaging agent or a pharmaceutically acceptable salt thereof; and—obtaining a dosage composition from the aqueous mixture which comprises the hyperpolarized biorelevant imaging agent or a pharmaceutically acceptable salt thereof.

Enumerated Embodiment 4. The method of Embodiment 3, wherein the method further comprises: —one or more washing steps, wherein the solution or aqueous mixture is washed with a second organic solvent, thereby forming: (i) an organic mixture phase comprising the first organic solvent, the second organic solvent, and optional unbonded sidearm, and (ii) an aqueous mixture phase comprising the aqueous mixture and the hyperpolarized biorelevant imaging agent or a pharmaceutically acceptable salt thereof; and—one or more separation steps, wherein the organic mixture phase is separated from the aqueous mixture phase, and either the organic mixture phase or the aqueous mixture phase is transferred into a separate vessel.

Enumerated Embodiment 5. The method of any one of Embodiments 1-4, wherein the step of obtaining a solution in a vessel comprises the following steps: —obtaining a solution comprising the first organic solvent and a biorelevant imaging agent precursor dissolved in the first organic solvent, wherein the biorelevant imaging agent precursor comprises: (i) a biorelevant imaging agent and a (ii) sidearm comprising an unsaturated carbon-carbon double bond (—C═C—) or carbon-carbon triple bond (—C≡C—); —hydrogenating the unsaturated carbon-carbon double bond (—C═C—) or carbon-carbon triple bond (—C≡C—) of the sidearm with parahydrogen in the first organic solvent, thereby forming a parahydrogenated derivative of the biorelevant imaging agent precursor; —applying a polarization transferring waveform to transfer nuclear spin order from the parahydrogen on the sidearm to a non-hydrogen nuclear spin on the biorelevant imaging agent; —optionally, hydrolyzing the parahydrogenated biorelevant imaging agent precursor by adding an aqueous hydrolyzing agent to the solution to produce the hyperpolarized biorelevant imaging agent and the unbonded sidearm; and—optionally neutralizing the solution with a buffer to slow or terminate the hydrolysis reaction; thereby producing the solution comprising the first organic solvent, an aqueous mixture optionally comprising the aqueous hydrolyzing agent, the hyperpolarized biorelevant imaging agent or a pharmaceutically acceptable salt thereof, and the optional unbonded sidearm.

Enumerated Embodiment 6. The method of Embodiment 5, wherein the step of obtaining a solution in a vessel further comprises a catalyst scavenging step prior to the addition of the aqueous hydrolyzing agent, wherein the catalyst scavenging step comprises filtering the solution to remove rhodium atoms, iridium atoms, and/or any other catalyst atoms from the solution; optionally wherein the solution is concentrated prior to the catalyst scavenging step.

Enumerated Embodiment 7. The method of any one of Embodiments 1 to 6, wherein the biorelevant imaging agent is selected from: pyruvate, glutamate, glutamine, lactate, acetate, acetoacetate, zymonate, alanine, fructose, fumarate, bicarbonate, urea, dehydroascorbate, alpha-ketoglutarate, dihydroxyacetone, glucose, ascorbate, and conjugate acids thereof.

Enumerated Embodiment 8. The method of Embodiment 7, wherein the biorelevant imaging agent has a solubility in the first organic solvent of less than 50 millimolar (mM).

Enumerated Embodiment 9. The method of Embodiment 7 or claim 8, wherein the biorelevant imaging agent has a solubility in water of greater than 50 millimolar (mM).

Enumerated Embodiment 10. The method of any one of Embodiments 1-9, wherein the aqueous mixture comprises: water, sodium hydroxide, potassium hydroxide, or any mixture thereof.

Enumerated Embodiment 11. The method of any one of claims 1-10, wherein the first organic solvent comprises acetone, ethanol, methanol, chloroform, ethyl acetate, methyl ethyl ketone (MEK), acetophenone, hexone, cyclohexanone, cyclopentanone, or a combination thereof.

Enumerated Embodiment 12. The method of Embodiment 11, wherein the first organic solvent comprises acetone.

Enumerated Embodiment 13. The method of Embodiment 11, wherein the first organic solvent comprises methyl ethyl ketone (MEK).

Enumerated Embodiment 14 The method of any one of Embodiments 1-13, wherein the first organic solvent has a solubility in water of greater than 75 millimolar (mM) at 20° C.

Enumerated Embodiment 15. The method of any one of Embodiments 1-14, wherein the second organic solvent comprises one or more ICH class 2 solvents selected from: acetonitrile, chlorobenzene, chloroform, cyclohexane, dibromomethane (DBM), 1,2-dichloroethene, dichloromethane (DCM), 1,2-dimethoxyethane, n,n-dimethylacetamide, n,n-dimethylformamide, 1,4-dioxane, 2-ethoxyethanol, ethyleneglycol, formamide, hexane, methanol, 2-methoxyethanol, methylbutyl ketone, methylcyclohexane, n-methylpyrrolidone, nitromethane, pyridine, sulfolane, tetrahydrofuran, tetralin, toluene, 1,1,2-trichloroethene, or xylene.

Enumerated Embodiment 16. The method of any one of Embodiments 1-14, wherein the second organic solvent comprises one or more ICH class 3 solvents selected from: acetic acid, acetone, anisole, 1-butanol, 2-butanol, butyl acetate, methyl-tert-butyl ether (MTBE), cumene, diethyl ether, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, or propyl acetate.

Enumerated Embodiment 17. The method of any one of Embodiments 1-16, wherein the second organic solvent comprises methyl-tert-butyl ether (MTBE).

Enumerated Embodiment 18. The method of any one of Embodiments 1-16, wherein the second organic solvent comprises dibromomethane (DBM).

Enumerated Embodiment 19. The method of any one of Embodiments 1-16, wherein the second organic solvent comprises dichloromethane (DCM).

Enumerated Embodiment 20. The method of any one of Embodiments 1-19, wherein the washing step and/or evaporation step is repeated until the first organic solvent has a concentration of 5000 ppm or less in the solution.

Enumerated Embodiment 21. The method of any one of Embodiments 1-20, wherein the washing step and/or evaporation step is repeated until the second organic solvent has a concentration in the solution below the ICH toxicity limit.

Enumerated Embodiment 22. The method of any one of Embodiments 1-22, wherein the evaporation step comprises bubbling with an inert gas; optionally wherein the inert gas is nitrogen gas.

Enumerated Embodiment 23. A dosage composition comprising hyperpolarized biorelevant imaging agent or a pharmaceutically acceptable salt thereof, wherein the dosage composition is produced by the method of any one of Embodiments 1-22.

Enumerated Embodiment 24. The dosage composition of Embodiment 23, comprising no more than 20 mM, 10 mM, 5 mM, 2 mM, or 1 mM of the first organic solvent.

Enumerated Embodiment 25. The dosage composition of Embodiment 23, comprising no more than 20 mM, 10 mM, 5 mM, 2 mM, or 1 mM of the second organic solvent.

Enumerated Embodiment 26. A system for implementing the method of any one of Embodiments 1-22.

Enumerated Embodiment 27. The system of Embodiment 26, comprising a first vessel, a second vessel fluidly connected with the first vessel through a first fluid transfer element, and optionally a third vessel fluidly connected with the second vessel through a second fluid transfer element.

Enumerated Embodiment 28. The system of Embodiment 26 or Embodiment 27, further comprising a magnetic guide system which provides a defined magnetic field over at least a portion of the system; optionally wherein the magnetic guide system provides a defined magnetic field over the entire system.

Enumerated Embodiment 29. The system of Embodiment 27, further comprising a magnetic guide system which provides a defined magnetic field over the first fluid transfer element and optional second fluid transfer element.

Enumerated Embodiment 30. The system of Embodiment 28 or Embodiment 29, wherein the defined magnetic field prevents undesired hyperpolarization loss in the solution as it is processed through the system.

### EXAMPLES

### Example 1-System for Liquid-Liquid Purification

A system was prepared comprising a first vessel, a second vessel fluidly connected to the first vessel, and a third vessel fluidly connected to the second vessel. The vessels and the fluid path were covered by a defined magnetic field provided by solenoids to prevent undesired hyperpolarisation loss during the processing of a solution through the system.

### Vessel 1

2.3-2.7 mL of a mixture comprising parahydrogenated and hyperpolarized pyruvate ester (with sidearm) in acetone (concentration between 180-250 mM) was injected into the first vessel. The mixture was bubbled with nitrogen gas, and 1.0 mL of base (400 mM NaOD in D2O) was injected into Vessel 1 to cleave the pyruvate ester into sodium pyruvate and the corresponding unbonded sidearm. 3 mL of phosphate buffer (18 mM NaH2PO4, 9 mM Na2HPO4, 52 mM NaCl in D2O) was then added to the mixture (while continuing to bubble with nitrogen gas). A sample was extracted and analyzed, and found to include a mixture of acetone, D2O, and sodium pyruvate (which was dissolved at a pH between 6-9).

The solution in Vessel 1 was then subjected to a first washing step. 20 mL of organic washing solvent (e.g., diethyl ether or tert-butyl methyl ether) were injected into the solution, and then agitated using nitrogen gas bubbling. The mixture was then allowed to settle for about 10 seconds until a clear phase separation between the aqueous phase (lower phase) and the organic phase (upper phase) had formed. Samples of each phase were extracted and analyzed: (i) most of the acetone, diethyl ether, and nonpolar side-products (including the unbound sidearm) were included in the organic phase; (ii) the sodium pyruvate remained in the aqueous phase (between 70-100 mM). The aqueous phase was also found to include residual concentrations of acetone (between 2-3.5 M) and diethyl ether (between 0.8-1 M).

### Vessel 2

The aqueous phase from Vessel 1 was pumped into Vessel 2, which had been prefilled with 20 mL of the same organic washing solvent (e.g., diethyl ether or tert-butyl methyl ether). The mixture was again agitated using nitrogen gas bubbling, and allowed to settle for about 10 seconds until a clear phase separation between the aqueous phase (lower phase) and the organic phase (upper phase) had formed. Samples of each phase were extracted and analyzed: (i) most of the acetone and diethyl ether were included in the organic phase; (ii) the sodium pyruvate remained in the aqueous phase, and the residual concentrations of acetone (between 300-700 mM) and diethyl ether (between 700-900 mM) were reduced.

### Vessel 3

Between 0.3-3 mL of the aqueous phase from Vessel 2 was pumped into Vessel 3. The solution is flushed with nitrogen gas via a frit to evaporate the residual volatile organics in the solution. The aqueous phase from Vessel 2 continues to be pumped into Vessel 3 as the solution continues to be exposed to volatile organic evaporation conditions with nitrogen gas flushing via a frit. Final samples were extracted and analyzed, and found to include the following: (i) sodium pyruvate (70-100 mM), acetone (200-400 mM), and diethyl ether (10-50 mM); pH was between 6-9.

### Example 2-Liquid-Liquid Purification Study

The liquid-liquid purification process was completed using parahydrogenated and hyperpolarized pyruvate ester with sidearm (200 mM start concentration) and the general system and processing steps of Example 1. Hydrolysis was completed using inline saponification with 200 mM base in a T-mixer and subsequent phosphate buffer to quench the hydrolysis reaction. Two washing steps were completed in 50 ml Falcon tubes (mixed by hand). Wash step 1 included an organic to aqueous ratio of 4:1. Wash step 2 included an organic to aqueous ratio of 6:1. Evaporation in Vessel 3 was completed by nitrogen gas bubbling at 1.5 L/min using glass frit at 60° C. and 150 mbar absolute pressure by vacuum.

Solvent combinations, specific processing conditions, and study results are shown in Table 2:

TABLE 2
Liquid-liquid purification parameters

| | | | Final | Final |
|---|---|---|---|---|
| | | Final | acetone/ | MTBE/ |
| | | pyruvate | MEK | DCM |
| First | Second | concen- | concen- | concen- |
| Organic | Organic | tration | tration | tration |
| Solvent | Solvent | (mM) | (mM) | (mM) |
| Acetone | methyl-tert-butyl | 178.1 | 2.01 | Not detectable |
| | ether (MTBE) | | | |
| methyl ethyl | methyl-tert-butyl | 63 | 16 | Not detectable |
| ketone (MEK) | ether (MTBE) | | | |
| methyl ethyl | dichloromethane | 138 | 10 | Not detectable |
| ketone (MEK) | (DCM) | | | |

Study results showed that the liquid-liquid purification system and processing steps were effective at producing a hyperpolarized pyruvate dosage composition with an ICH-acceptable concentration of organic solvents, using either acetone or MEK as the first organic solvent, and MTBE or DCM as the second organic solvent.

### Example 3-Acetone Extraction Study

A study was completed to analyze the effectiveness of extracting acetone from an aqueous phase using five different organic washing solvents: MTBE (methyl-tert-butyl ether); DCM (dichloromethane); DBM (dibromoethane); Et₂O (diethylether); and DCB (dichlorobenzene). Extraction efficiency for each solvent was tested at two different acetone concentrations (9000 mM and 2000 mM), both with and without pyruvate added to each concentration, and with an organic wash:aqueous volume ratio of 4:1. Extraction efficiency was calculated as output acetone concentration in water (after washing) divided by input acetone concentration in water (before washing), with acetone concentration being measured by NMR.

Study results (extraction efficiency) are shown in Table 3:

TABLE 3
Acetone extraction study results

| Washing | 9000 mM acetone | | 2000 mM acetone | |
|---|---|---|---|---|
| Solvent | No Pyruvate | With Pyruvate | No Pyruvate | With Pyruvate |
| MTBE | 0.24 | 0.23 | 0.33 | 0.27 |
| DCM | 0.07 | 0.06 | — | 0.04 |
| DBM | — | 0.09 | — | 0.07 |
| Et₂O | 0.29 | — | — | — |
| DCB | 0.25 | — | — | — |

Study results showed DCM (dichloromethane) and DBM (dibromoethane) as having the best extraction efficiency (lowest output/input ratio), both with and without pyruvate in solution. MTBE, Et₂O, and DCB were also shown to have effective extraction efficiency, though less effective (higher output/input ratio) compared to DCM and DBM.

### Example 4-Evaporation Study

A study was completed to analyze the effectiveness of using evaporation to remove residual organic solvent materials from water. Evaporation conditions were similar to those in Example 2 (nitrogen gas bubbling at 1.5 L/min using glass frit at 60° C. and 150 mbar absolute pressure by vacuum). The following solutions were including for study: Study results (extraction efficiency) are shown in Table 3: (i) Mixture 1—466 mM Acetone+230 mM MTBE in water; (ii) Mixture 2—466 mM Acetone+150 mM DCM in water; (ii) Mixture 3—277 mM Acetone+332 mM MTBE in water. Extraction efficiency was calculated as output acetone concentration in water (after evaporation) divided by input acetone concentration in water (before evaporation), with acetone concentration being measured by NMR.

Evaporation of Mixture 1 produced an acetone extraction efficiency of 0.070.

Evaporation of Mixture 2 produced an acetone extraction efficiency of 0.040.

Evaporation of Mixture 3 produced an acetone extraction efficiency of 0.086.

Study results showed that the evaporation process and parameters of the liquid-liquid purification system is effective at extracting the residual organic solvent from an aqueous mixture at varying concentrations of acetone and MTBE/DCM.

### Example 5-SABRE Catalyst in Acetone Extraction Study

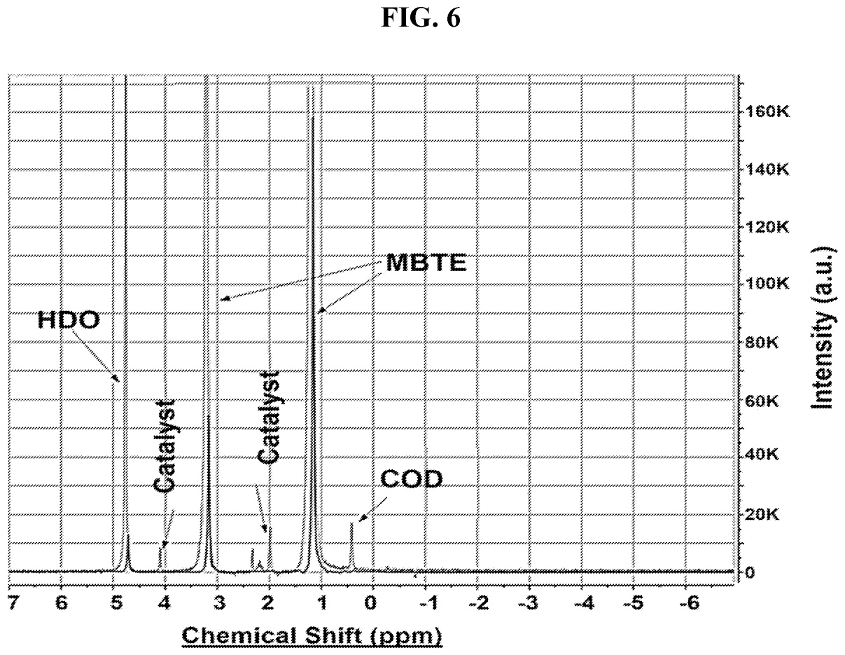

5 mg of IrIMes(COD) PF6 precatalyst was dissolved in 1 ml Acetone-d6. The solution was subsequently mixed with 6 ml of MTBE and 1 ml of D2O. The water and organic phase separated within seconds, and both were extracted for proton NMR measurements. The concentration of both the acetone and the precatalyst was lower in the aqueous phase after washing. The NMR spectrum for both phases is shown in FIG. 6. As can be seen in the NMR spectrum, the precatalyst was observable only in one of the phases and not in the other.

What is claimed is:

1. A method for producing a dosage composition comprising a hyperpolarized biorelevant imaging agent or a pharmaceutically acceptable salt thereof, the method comprising:
obtaining a solution in a vessel, wherein the solution comprises a first organic solvent, an aqueous mixture, the hyperpolarized biorelevant imaging agent or a pharmaceutically acceptable salt thereof, and optionally an unbonded sidearm;
one or more washing steps, wherein the solution is washed with a second organic solvent, thereby forming: (i) an organic mixture phase comprising the first organic solvent, the second organic solvent, and the optional unbonded sidearm, and (ii) an aqueous mixture phase comprising the aqueous mixture and the hyperpolarized biorelevant imaging agent or a pharmaceutically acceptable salt thereof;
one or more separation steps wherein the organic mixture phase is separated from the aqueous mixture phase, and either the organic mixture phase or the aqueous mixture phase is transferred into a separate vessel; and
obtaining a dosage composition from the aqueous mixture which comprises the hyperpolarized biorelevant imaging agent or a pharmaceutically acceptable salt thereof.

2. The method of claim 1, wherein the method further comprises one or more evaporation steps, wherein the aqueous mixture phase is subjected to organic vapor extraction conditions to evaporate at least a portion of organic solvent remaining in the aqueous mixture.

3. A method for producing a dosage composition comprising a hyperpolarized biorelevant imaging agent or a pharmaceutically acceptable salt thereof, the method comprising:
obtaining a solution in a vessel, wherein the solution comprises a first organic solvent, an aqueous mixture, the hyperpolarized biorelevant imaging agent or a pharmaceutically acceptable salt thereof, and optionally an unbonded sidearm;
one or more evaporation steps, wherein the solution is subjected to organic vapor extraction conditions to evaporate a portion of organic solvent, and optional unbonded sidearm, from the solution, thereby providing an aqueous mixture phase comprising the aqueous mixture and the hyperpolarized biorelevant imaging agent or a pharmaceutically acceptable salt thereof; and
obtaining a dosage composition from the aqueous mixture which comprises the hyperpolarized biorelevant imaging agent or a pharmaceutically acceptable salt thereof.

4. The method of claim 3, wherein the method further comprises:
one or more washing steps, wherein the solution or aqueous mixture is washed with a second organic solvent, thereby forming: (i) an organic mixture phase comprising the first organic solvent, the second organic solvent, and optional unbonded sidearm, and (ii) an aqueous mixture phase comprising the aqueous mixture and the hyperpolarized biorelevant imaging agent or a pharmaceutically acceptable salt thereof; and
one or more separation steps, wherein the organic mixture phase is separated from the aqueous mixture phase, and either the organic mixture phase or the aqueous mixture phase is transferred into a separate vessel.

5. The method of any one of claims 1-4, wherein the step of obtaining a solution in a vessel comprises the following steps:
obtaining a solution comprising the first organic solvent and a biorelevant imaging agent precursor dissolved in the first organic solvent, wherein the biorelevant imaging agent precursor comprises: (i) a biorelevant imaging agent and a (ii) sidearm comprising an unsaturated carbon-carbon double bond (—C═C—) or carbon-carbon triple bond (—C≡C—);
hydrogenating the unsaturated carbon-carbon double bond (—C═C—) or carbon-carbon triple bond (—C≡C—) of the sidearm with parahydrogen in the first organic solvent, thereby forming a parahydrogenated derivative of the biorelevant imaging agent precursor;
applying a polarization transferring waveform to transfer nuclear spin order from the parahydrogen on the sidearm to a non-hydrogen nuclear spin on the biorelevant imaging agent;
optionally, hydrolyzing the parahydrogenated biorelevant imaging agent precursor by adding an aqueous hydrolyzing agent to the solution to produce the hyperpolarized biorelevant imaging agent and the unbonded sidearm; and
optionally neutralizing the solution with a buffer to slow or terminate the hydrolysis reaction;
thereby producing the solution comprising the first organic solvent, an aqueous mixture optionally comprising the aqueous hydrolyzing agent, the hyperpolarized biorelevant imaging agent or a pharmaceutically acceptable salt thereof, and the optional unbonded sidearm.

6. The method of claim 5, wherein the step of obtaining a solution in a vessel further comprises a catalyst scavenging step prior to the addition of the aqueous hydrolyzing agent, wherein the catalyst scavenging step comprises filtering the solution to remove rhodium atoms, iridium atoms, and/or any other catalyst atoms from the solution; optionally wherein the solution is concentrated prior to the catalyst scavenging step.

7. The method of any one of claims 1 to 6, wherein the biorelevant imaging agent is selected from: pyruvate, glutamate, glutamine, lactate, acetate, acetoacetate, zymonate, alanine, fructose, fumarate, bicarbonate, urea, dehydroascorbate, alpha-ketoglutarate, dihydroxyacetone, glucose, ascorbate, and conjugate acids thereof.

8. The method of claim 7, wherein the biorelevant imaging agent has a solubility in the first organic solvent of less than 50 millimolar (mM).

9. The method of claim 7 or claim 8, wherein the biorelevant imaging agent has a solubility in water of greater than 50 millimolar (mM).

10. The method of any one of claims 1-9, wherein the aqueous mixture comprises: water, sodium hydroxide, potassium hydroxide, or any mixture thereof.

11. The method of any one of claims 1-10, wherein the first organic solvent comprises acetone, ethanol, methanol, chloroform, ethyl acetate, methyl ethyl ketone (MEK), acetophenone, hexone, cyclohexanone, cyclopentanone, or a combination thereof.

12. The method of claim 11, wherein the first organic solvent comprises acetone.

13. The method of claim 11, wherein the first organic solvent comprises methyl ethyl ketone (MEK).

14. The method of any one of claims 1-13, wherein the first organic solvent has a solubility in water of greater than 75 millimolar (mM) at 20° C.

15. The method of any one of claims 1-14, wherein the second organic solvent comprises one or more ICH class 2 solvents selected from: acetonitrile, chlorobenzene, chloroform, cyclohexane, dibromomethane (DBM), 1,2-dichloroethene, dichloromethane (DCM), 1,2-dimethoxyethane, n,n-dimethylacetamide, n,n-dimethylformamide, 1,4-dioxane, 2-ethoxyethanol, ethyleneglycol, formamide, hexane, methanol, 2-methoxyethanol, methylbutyl ketone, methylcyclohexane, n-methylpyrrolidone, nitromethane, pyridine, sulfolane, tetrahydrofuran, tetralin, toluene, 1,1,2-trichloroethene, or xylene.

16. The method of any one of claims 1-14, wherein the second organic solvent comprises one or more ICH class 3 solvents selected from: acetic acid, acetone, anisole, 1-butanol, 2-butanol, butyl acetate, methyl-tert-butyl ether (MTBE), cumene, diethyl ether, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, or propyl acetate.

17. The method of any one of claims 1-16, wherein the second organic solvent comprises methyl-tert-butyl ether (MTBE).

18. The method of any one of claims 1-16, wherein the second organic solvent comprises dibromomethane (DBM).

19. The method of any one of claims 1-16, wherein the second organic solvent comprises dichloromethane (DCM).

20. The method of any one of claims 1-19, wherein the washing step and/or evaporation step is repeated until the first organic solvent has a concentration of 5000 ppm or less in the solution.

21. The method of any one of claims 1-20, wherein the washing step and/or evaporation step is repeated until the second organic solvent has a concentration in the solution below the ICH toxicity limit.

22. The method of any one of claims 1-22, wherein the evaporation step comprises bubbling with an inert gas; optionally wherein the inert gas is nitrogen gas.

23. A dosage composition comprising hyperpolarized biorelevant imaging agent or a pharmaceutically acceptable salt thereof, wherein the dosage composition is produced by the method of any one of claims 1-22.

24. The dosage composition of claim 23, comprising no more than 20 mM, 10 mM, 5 mM, 2 mM, or 1 mM of the first organic solvent.

25. The dosage composition of claim 23, comprising no more than 20 mM, 10 mM, 5 mM, 2 mM, or 1 mM of the second organic solvent.

26. A system for implementing the method of any one of claims 1-22.

27. The system of claim 26, comprising a first vessel, a second vessel fluidly connected with the first vessel through a first fluid transfer element, and optionally a third vessel fluidly connected with the second vessel through a second fluid transfer element.

28. The system of claim 26 or claim 27, further comprising a magnetic guide system which provides a defined magnetic field over at least a portion of the system; optionally wherein the magnetic guide system provides a defined magnetic field over the entire system.

29. The system of claim 27, further comprising a magnetic guide system which provides a defined magnetic field over the first fluid transfer element and optional second fluid transfer element.

30. The system of claim 28 or claim 29, wherein the defined magnetic field prevents undesired hyperpolarization loss in the solution as it is processed through the system.
